(19) United States
(12) Patent Application Publication
(10) Pub. No.: US 2007/0141106 A1
(43) Pub. Date: Jun. 21, 2007

(54) DRUG ELUTING IMPLANT

(76) Inventors: Peter M. Bonutti, Effingham, IL (US); Matthew J. Cremens, Effingham, IL (US)

Correspondence Address: PAUL D. BIANCO: FLEIT, KAIN, GIBBONS,;GUTMAN, BONGINI, & BIANCO P.L., 21355 EAST DIXIE HIGHWAY, SUITE 115, MIAMI, FL, 33180, US

(21) Appl. No.: 11/549,994

(22) Filed: Oct. 17, 2006

Related U.S. Application Data
(60) Provisional application No. 60/728,206, filed on Oct. 19, 2005.

(51) Int. Cl. A61N 1/30; A61F 2/02

(52) U.S. Cl. 424/423; 604/20

(57) ABSTRACT

The present invention provides a medical system for the administration of a pharmaceutical agent in vivo to a patient. The medical system includes a medical implant positionable in a body of a patient. A pharmaceutical agent in disposed on the medical implant and at least partially coated with a reactive coating. The reactive coating act to controls the release of the pharmaceutical agent. An energy unit is provided for transmitting an energy signal to the reactive coating, wherein the reactive coating reacts to the energy signal to increase the release rate of the pharmaceutical agent.

### CROSS-REFERENCE TO RELATED APPLICATION

This application claims priority to U.S. Provisional Patent Application No. 60/728,205, entitled Drug Eluting Implant, filed on Oct. 19, 205, the contents of which are incorporated by references in there entirety.

### FIELD OF THE INVENTION

The present invention relates to a method and device for controlling the release of one or more pharmaceutical agents in a localized area of a body of a patient. In particular, the present invention relates to an implantable medical device having a chamber or coating for controlling the release of a pharmaceutical agent.

### BACKGROUND OF THE INVENTION

Accurate delivery of small, precise quantities of one or more therapeutic or medicinal agents to a localized area of a body of a patient is of great importance in many different fields of science and industry. To accomplish this, it is generally known to provide a coating including therapeutic or medicinal agents on an implantable medical device. Alternatively, it is also generally known to provide an implantable device having a reservoir for the therapeutic or medical agents. Upon insertion into the body of the patient, the therapeutic or medicinal agents are released from the implantable medical device into the localized area.

The controlled release of therapeutic or medicinal agents can utilize various technologies. Devices are known having a monolithic layer or coating incorporating a heterogeneous solution and/or dispersion of an active agent in a polymeric substance, where the diffusion of the agent is rate limiting, as the agent diffuses through the polymer to the polymer-fluid interface and is released into the surrounding fluid. In some devices, a soluble substance is also dissolved or dispersed in the polymeric material, such that additional pores or channels are left after the material dissolves. A matrix device is generally diffusion limited as well, but with the channels or other internal geometry of the device also playing a role in releasing the agent to the fluid. The channels can be pre-existing channels or channels left behind by released agent or other soluble substances.

Erodible or degradable devices typically have the active agent physically immobilized in the polymer. The active agent can be dissolved and/or dispersed throughout the polymeric material. The polymeric material is often hydrolytically degraded over time through hydrolysis of labile bonds, allowing the polymer to erode into the fluid, releasing the active agent into the fluid. Hydrophilic polymers have a generally faster rate of erosion relative to hydrophobic polymers. Hydrophobic polymers are believed to have almost purely surface diffusion of active agents, having erosion from the surface inwards. Hydrophilic polymers are believed to allow water to penetrate the surface of the polymer, allowing hydrolysis of labile bonds beneath the surface, which can lead to homogeneous or bulk erosion of the polymer.

A common characteristic of these agent-coated and agent-loaded implantable medical devices is that the dissolving or eluting mechanism of the agents is not controllable or selectable by the medical practitioner. The agent coating or loading is designed to release the agents at a set time, together with conditions within the patient, which causes the agents to be delivered in a manner that cannot be controlled or selected once the coated or loaded implantable device is positioned in the body of the patient. Thus, the agent effect will continue to run its course even if the underlying reasons for the agent are no longer present. For example, if an agent is designed to have an inhibiting effect on tissue growth, that effect may go too far and actually be deleterious to the tissue.

Thus, these exists a need for an improved drug eluting implant.

### SUMMARY OF THE INVENTION

The present invention provides a medical system for the administration of a pharmaceutical agent in vivo to a patient. The medical system includes a medical implant positionable in a body of a patient. A pharmaceutical agent in disposed on the medical implant and is at least partially coated with a reactive coating. The reactive coating acts to control the release of the pharmaceutical agent. An energy unit may be provided for transmitting an energy signal to the reactive coating, wherein the reactive coating reacts to the energy signal to increase the release rate of the pharmaceutical agent.

The reactive coating may be a porous coating, including a plurality of pores. The pores increase in size in response to the energy signal, increasing the release rate of the pharmaceutical agent. Alternatively, the reactive coating may be a biodegradable coating. The energy signal increases the degradation rate of the biodegradable coating, increasing the release rate of the pharmaceutical agent.

In one embodiment, the medical implant is made of a biodegradable material and includes the pharmaceutical agent therein. The degradation rate of the biodegradable medical implant may be increased in response to the energy signal. The increased degradation rate increases the release rate of the pharmaceutical agent. The biodegradable medical implant may be made up of a plurality of biodegradable layers, wherein each of the layers includes the pharmaceutical agent there between or therein. The energy signal may be used to selectively remove a layer of the biodegradable medical implant, increasing the release rate of the pharmaceutical agent therein.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 depicts a medical implant of the present invention including at least one pharmaceutical agent thereon;

FIG. 2 depicts a medical implant of the present invention including three layers of pharmaceutical agents thereon;

FIG. 3 depicts another embodiment of the medical implant of the present invention including a polymer coating;

FIG. 4 depicts the medical implant of FIG. 3 including multiple polymer coatings;

FIG. 5A-B depict medical implants of FIG. 3 including porous coatings;

FIG. 6 depicts the medical implant of FIG. 3 including a biodegradable coating;

FIG. 7 depicts the medical implant of FIG. 3 including a micro capsule coating;

FIGS. 5A-B depict the medical implant of FIG. 3 including reservoirs for receiving pharmaceutical agents;

FIG. 9 depicts an energy unit in use with the medical implant of the present invention;

FIG. 10 depicts a schematic diagram of an energy unit according to present invention utilizing acoustic waves;

FIG. 11 depicts a biodegradable medical implant of the present invention impregnated with a pharmaceutical agent;

FIG. 12 depicts a cross sectional view of the biodegradable medical implant of the present invention including multiple layers;

FIG. 13 depicts an energy unit of the present invention being inserted through an expandable cannula;

FIG. 14 depicts an internal energy unit of the present invention including a power supply;

FIG. 15 depicts a rechargeable power supply for the internal energy unit of FIG. 14;

FIG. 16 depicts another embodiment of the energy unit of FIG. 14 including a control unit;

FIG. 17 depicts the medical implant of FIG. 3 including a non-degradable coating;

FIG. 18 depicts the non-reabsorbable coating of FIG. 17 in a cracked configuration;

FIG. 19 depicts a medical implant of the present invention including an energy sink;

FIG. 20 depicts a magnetically or electrically charged medical implant of the present invention;

FIG. 21 depicts a medical implant of the present invention including coverable reservoirs;

FIG. 22 depicts a cover portion of varying thickness for the medical implant of FIG. 21;

FIG. 23 depicts an alternative embodiment of the medical implant of FIG. 21;

FIG. 24 depicts a medical implant of the present invention including a coverable cavity;

FIG. 25 depicts the medical implant of FIG. 24 including a plurality of coverable cavities;

FIG. 26 depicts an alternative medical implant of the present invention including a coverable cavity;

FIG. 27 depicts the medical implant of FIG. 26 including a plurality of coverable cavities;

FIG. 28 depicts the medical implant of FIGS. 21-27 including an absorbent substrate;

FIG. 29 depicts the medical implant of FIGS. 21-27 used in conjunction with a suture to secure body tissue;

FIG. 30 depicts a mesh material of the present invention;

FIG. 31 depicts the mesh material of FIG. 30, including a pharmaceutical agent thereon;

FIG. 32 depicts the mesh material of FIG. 30 formed into a mesh band for positioning about a vessel in the body of a patient;

FIG. 33 depicts the mesh material of FIG. 30 formed into a mesh band for positioning partially about a heart in the body of a patient;

FIG. 34 depicts the mesh material of FIG. 30 formed into a mesh pouch configured for receiving an agent;

FIG. 35 depicts the mesh material of FIG. 30 positioned about a medical implant;

FIG. 36 depicts an exploded view of an alternative medical implant of the present invention including an internal cavity;

FIG. 37 depicts a front view of the medical implant of FIG. 36;

FIG. 38 depicts an isometric view of the implant of FIG. 36;

FIG. 39 depicts a front sectional view of the medical implant of FIG. 36;

FIG. 40 depicts an exemplary expandable cannula; and

FIG. 41 depicts an exemplary balloon dissection device.

### DETAILED DESCRIPTION OF THE INVENTION

The present invention provides a medical system for the administration of a pharmaceutical agent in vivo to a patient. The medical system includes a medical implant positionable in a body of a patient. A pharmaceutical agent is disposed on the medical implant and is at least partially coated with a reactive coating. As discussed in more detail below, the pharmaceutical agent can be any therapeutic substance and the reactive coating can be made of any suitable biocompatible material. Similarly, the medical implant is made from biocompatible materials such as metallic, polymeric, ceramic, and composite materials.

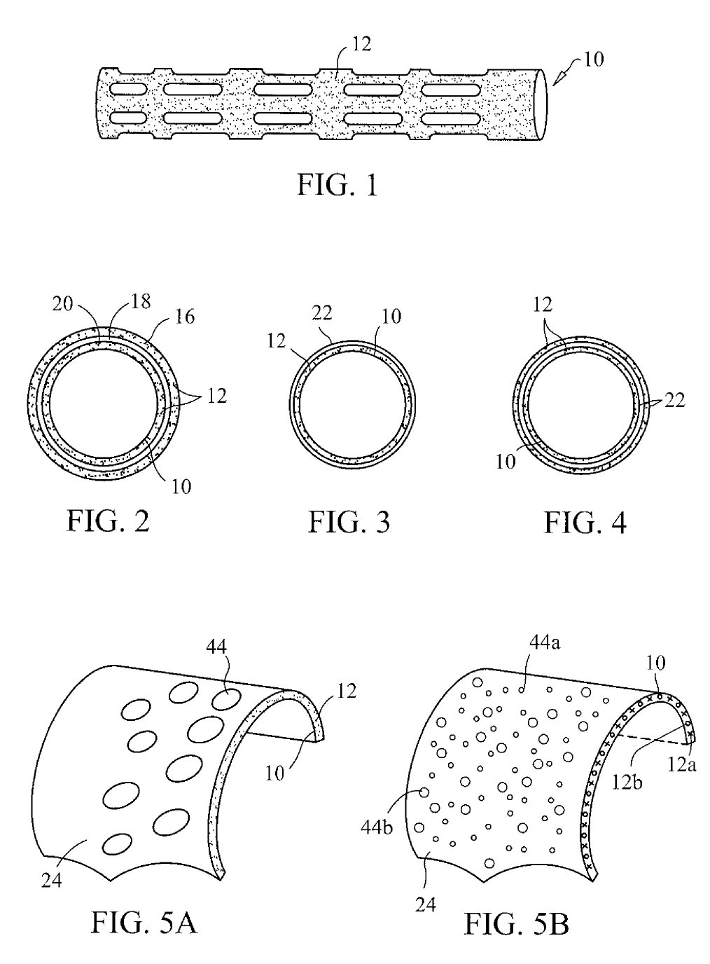

Referring now to the figures in which like reference numerals refer to like elements, a medical implant 10 according to the present invention is shown in FIG. 1. The medical implant 10 may be coated with a pharmaceutical agent 12. The pharmaceutical agent 12 being bonded to the surface of the medical implant 10 by, for example, but not limited to, covalent bonding, ionic bonding, VanderWal forces, magnetic forces, etc. A primer layer can be placed on the implant 10 and would be positioned between the implant 10 and the agent 12. A top coat could be placed over the agent 12. The medical implant 10 may include a single layer of a single or combination of pharmaceutical agents 12.

Alternatively, the medical implant 10 may include multiple layers of a single or a combination of pharmaceutical agents 12. Each of the multiple layers may contain the same pharmaceutical agents 12, having the same dosage.

It is further contemplated that the dosage of the pharmaceutical agents 12 (and/or the composition of the agents) in each of the multiple layers may be different. A treatment protocol may require that different dosages of the pharmaceutical agents 12 or different composition of the agents be released at different times during the treatment protocol. The multiple-layers, each containing different dosages of the pharmaceutical agents 12 or different compositions of the agents, allow for the controllable release of the differing agents during the protocol.

Referring to FIG. 2, the medical implant 10 may include three pharmaceutical agent layers: a top layer 16, a middle layer 18, and a bottom layer 20. The dosage of the pharmaceutical agent 12 in each of the layers 16, 18, and 20 is different, wherein the dosage of the pharmaceutical agent 12 decreases from the top layer 16 to the bottom layer 20. Alternatively, each of the multiple layers may contain a different pharmaceutical agent 12.

The pharmaceutical agent 12 may be, for example, a drug. Where the medical implant 10 is a stent, the drug may be used for the prevention or treatment of restenosis. Formulations useful for restenosis prevention or treatment can include, but are not limited to, heparin and heparin fragments, colchicine, taxol, agiotensin converting enzyme (ACE) inhibitors, angiopeptin, Cyclosporin A, goat-anti-rabbit PDGF antibody, terbinafine, trapidil, interferon-gamma, steroids, ionizing radiation, fusion toxins, antisense oligonucleotides, gene vectors, and rapamycin.

In addition to or as an alternative to, the pharmaceutical agent 12 may be a therapeutic biologic agent. Examples of such agents include, but are not limited to, hormones, cells, fetal cells, stem cells, bone morphogenic proteins (BMPs), tissue inductive factors, enzymes, proteins, RNA, viruses, etc.

Furthermore, the pharmaceutical agent 12 can be a binary agent, including a first and second compound. The first and second compounds beneficially interact to provide an increased tissue response. Each of the first and second compounds are separately disposed on the medical implant 10, upon release of which beneficially interact. Alternatively, a first compound is disposed on the medical implant 10, upon release of which the second compound is introduced into patient. The second compound can be introduces intravenously into the patient, traveling through the body of the patient to the treatment site. Alternatively, the second compound can be introduced directing into the treatment site, either through direct injection or surgical techniques.

Referring to FIG. 3, a pharmaceutical agent 12 may be affixed to the medical implant 10 by bonding the pharmaceutical agent 12 to the medical implant 10 and coating the medical implant 10 and pharmaceutical agent 12 with a polymer coat 22. The pharmaceutical agent 12 is released to the local treatment area by seeping through the polymer coating 22. The release rate of the pharmaceutical agent 12 is proportional to the thickness and/or permeability of the polymer coating 22.

Additionally, polymer coating 22 can be a degradable coating. The pharmaceutical agent 12 is initially released to the local treatment area by seeping through the polymer coating 22. As the polymer coating 22 degrades, the release rate of the pharmaceutical agent 12 may be increased.

In an embodiment, the medical implant 10 may include a gelatin substrate impregnated with the pharmaceutical agent 12. For example, the medical implant 10 is coated with the impregnated gelatin substrate and further coated with the polymer coat 22. The polymer coating 22 protects the integrity of the gelatin substrate, substantially preventing the release of the pharmaceutical agent. As the polymer coating degrades, the gelatin substrate is at least partially exposed to body fluids, releasing the pharmaceutical agent 12. The gelatin substrate may be beneficial in storing active biologic agents, such as fetal cells, stem cells, viruses, RNA, etc. Although any suitable matrix can be used, a gelatin substrate is believed to be particularly useful for certain agents. Upon the degradation of the polymer coating, the biologic agents seep from the gelatin substrate.

The polymer coating 22 can include, for example, polyurethanes, polyethylene terephthalate (PET), PLLA-poly-glycolic acid (PGA) copolymer (PLGA), polycaprolactone (PCL) poly-(hydroxybutyrate/hydroxyvalerate) copolymer (PHBV), poly(vinylpyrrolidone) (PVP), polytetrafluoroethylene (PTFE, Teflon®, poly(2-hydroxyethylmethacrylate) (poly-HEMA), poly(etherurethane urea), silicones, acrylics, epoxides, polyesters, urethanes, parlenes, polyphosphazene polymers, fluoropolymers, polyamides, polyolefins, and mixtures thereof.

Alternatively, the release rate and dosage of the pharmaceutical agent 12 may be controlled by covering only selected portions of the medical implant 10 and pharmaceutical agent 12. The uncovered portions of the medical implant 10 and pharmaceutical agent 12 will release at a greater rate than the covered positions of the medical implant 10 and pharmaceutical agent 12. In such instances, the partial polymer coating 22 may be used to vary the dosage and release rate of the pharmaceutical agent 12. For example, initially a greater dosage of the pharmaceutical agent 12 may be required, which may be provided by the uncovered portion of the pharmaceutical agent 12. At a later time period, a lesser dosage of the pharmaceutical agent 12 may be required, which may be provided by the covered portions of the medical implant 10 and pharmaceutical agent 12. Alternatively, the thickness of the polymer coating 22 may be varied to control the release rate of the pharmaceutical agent 12.

Referring to FIG. 4, the medical implant 10 may includes a plurality of polymer coatings 22, wherein a pharmaceutical agent 12 is disposed between each layer of the polymer coatings 22. Each of the layers may contain the same pharmaceutical agent 12, having the same dosage. Alternatively, the dosage of the pharmaceutical agent 12 between each of the multiple polymer coating 22 layers may be different. Additionally, each of the polymer coating 22 layers may contain a different pharmaceutical agent 12 there between.

The polymer coating 22 has been described as is at least partially covering the pharmaceutical agent 12. It is also contemplated that the pharmaceutical agent 12 may be mixed in or bonded to the polymer coating 22. The pharmaceutical agent 12 is released to the local treatment area by eluting from the polymer coating 22. The release rate of the pharmaceutical agent 12 is proportional to the concentration of the pharmaceutical agent 12 present in the polymer coating, to the thickness and/or permeability of the polymer coating 22.

Referring to FIG. 5 A, the polymer coating may be a porous minicellular coating 24. The porous coating 24 acts as a barrier limiting the release of the pharmaceutical agent 12, wherein the rate of diffusion of the pharmaceutical agent 12 is regulated by the size of the pores 44 in the coating 24. The porous coating 24 may be directly covering the pharmaceutical agent 12, or in the alternative, be used in conjunction with another polymer coating (porous or non-porous) to further control the release of the pharmaceutical agent 12.

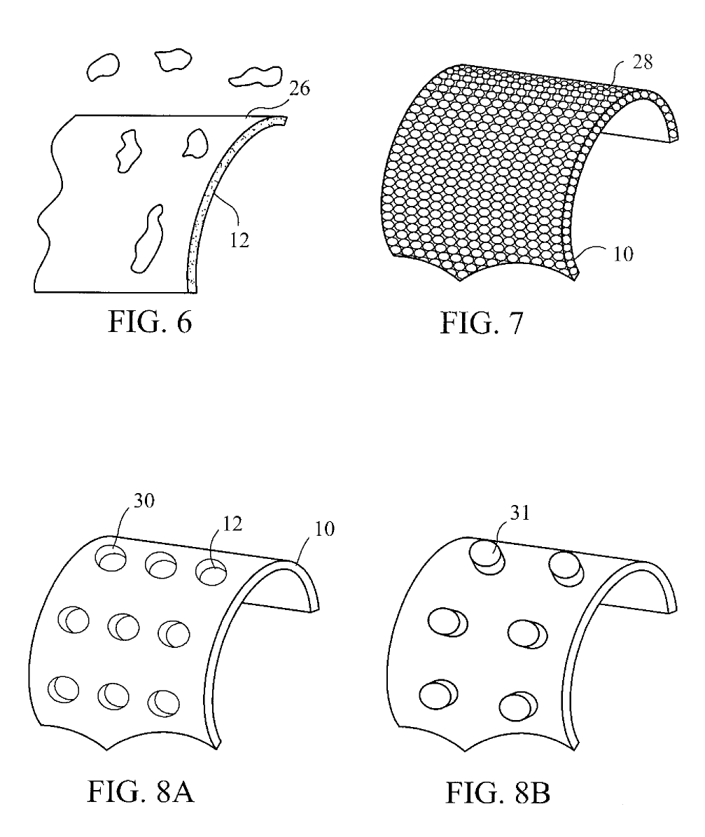

Referring to FIG. 6, the polymer coating may be a biodegradable coating 26. The biodegradable coating 26 may be used to control the release rate of the pharmaceutical agent 12. As the biodegradable coating 26 degrades, the pharmaceutical agent 12 is released. It is contemplated that the medical implant 10 may include multiple biodegradable coating 26 layers, wherein the biodegradable coating 26 layers each contain the same or a different pharmaceutical agent 12. As an upper biodegradable layer degrades, the pharmaceutical agent 12 therein is released, exposing a lower layer biodegradable layer. The lower layer will then begin to degrade, releasing the pharmaceutical agent 12 therein.

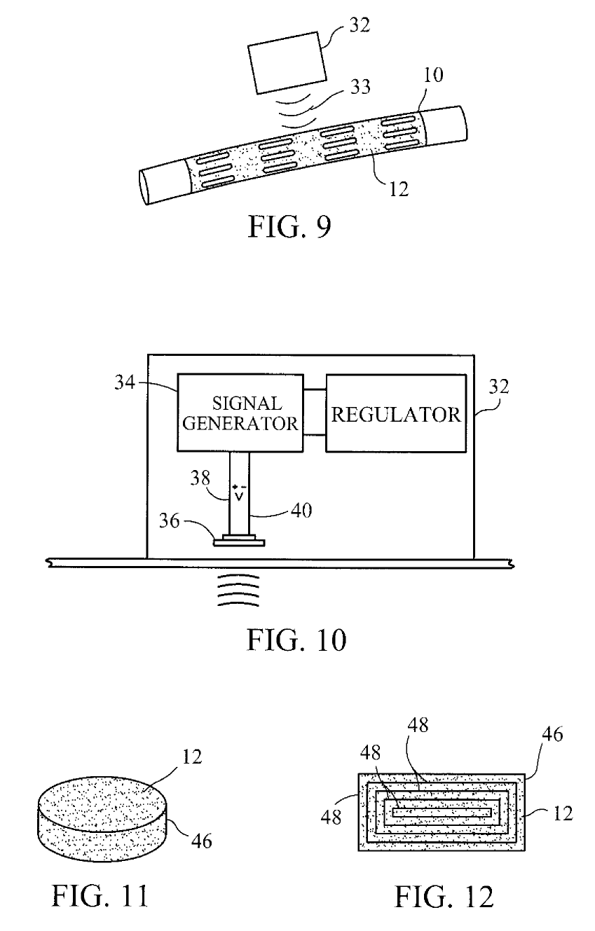

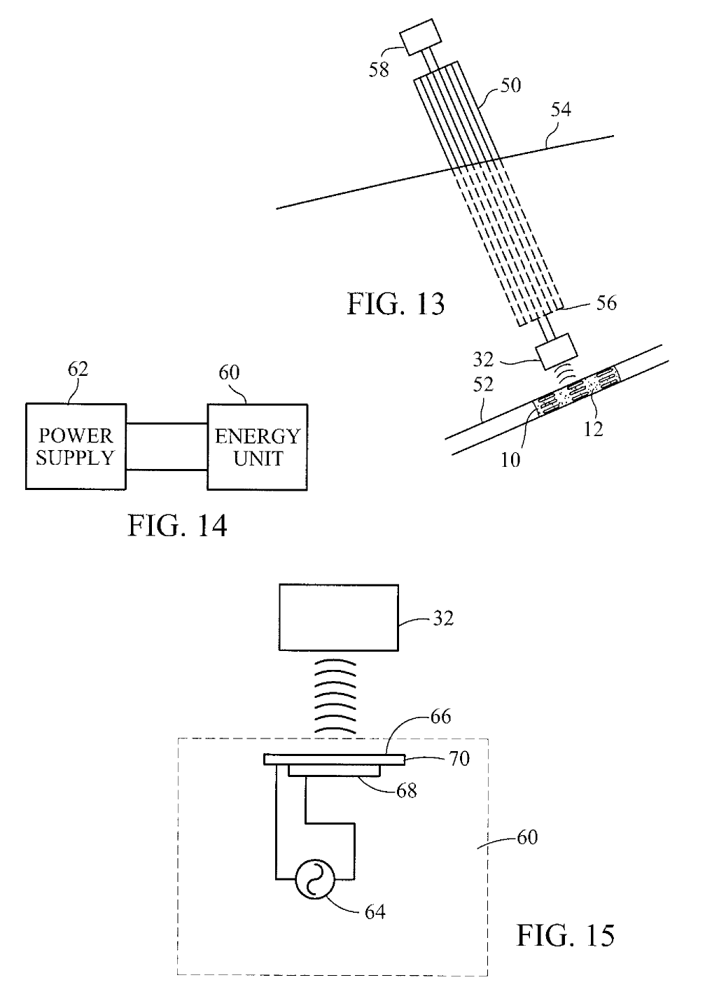

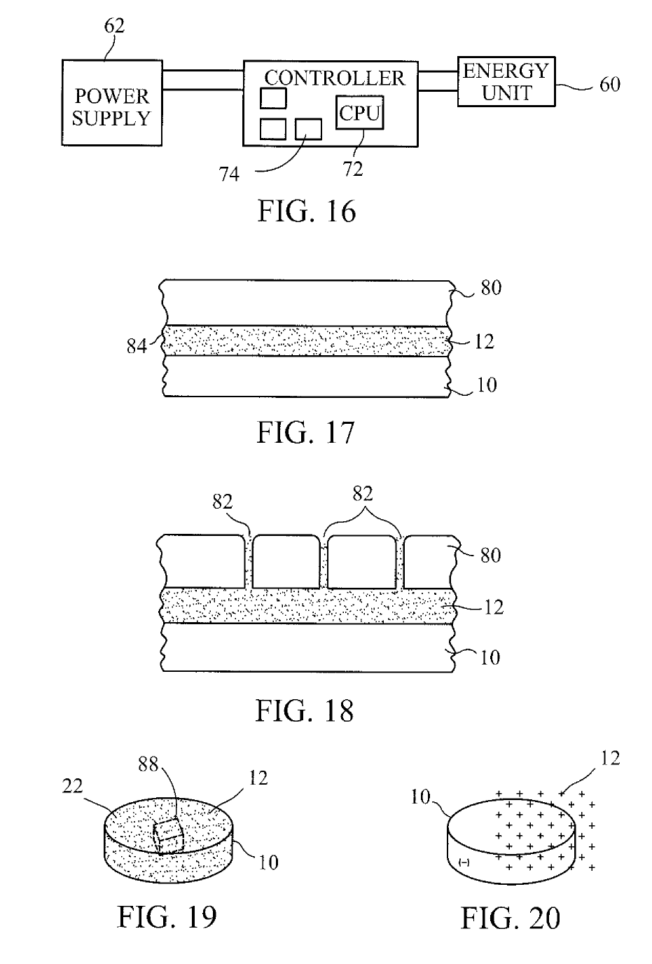

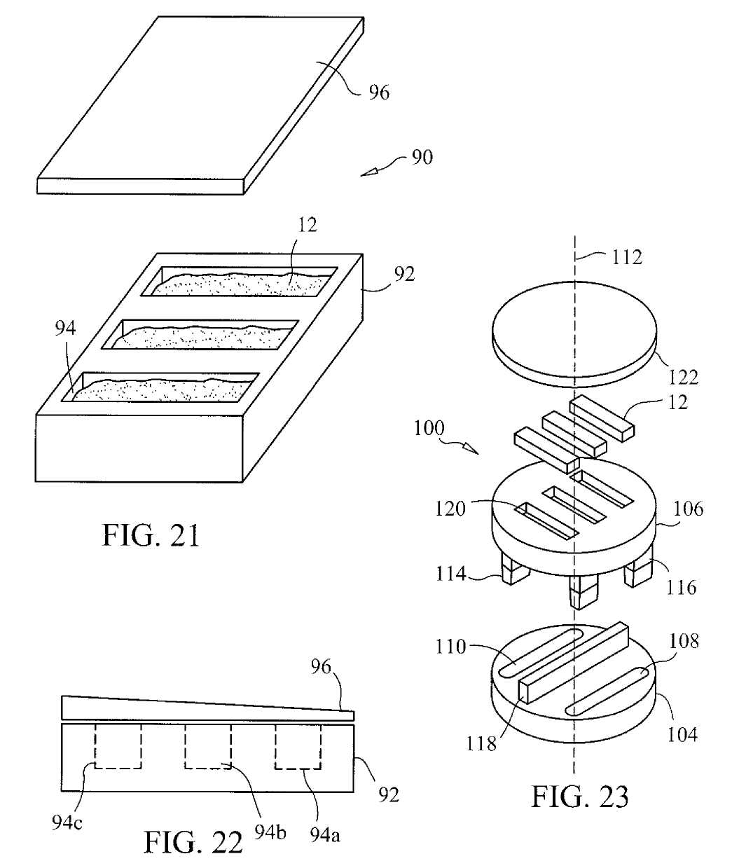

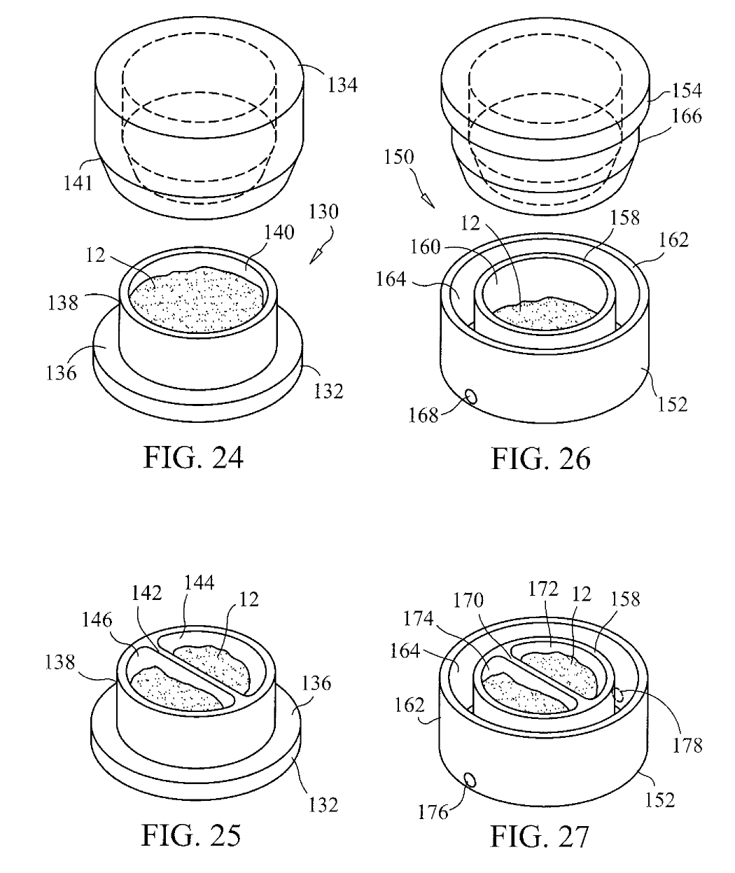

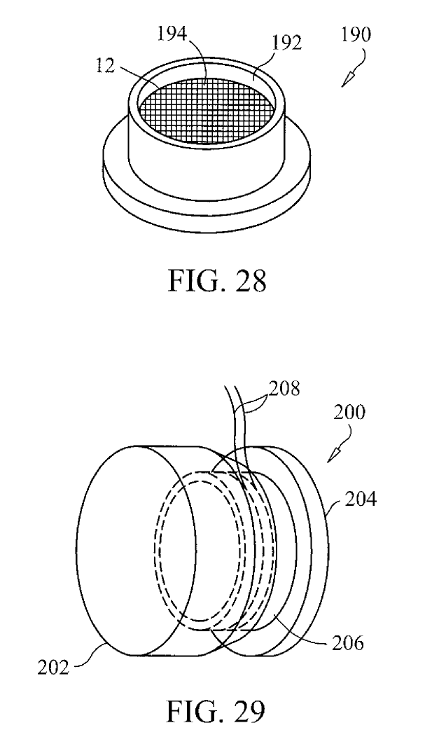

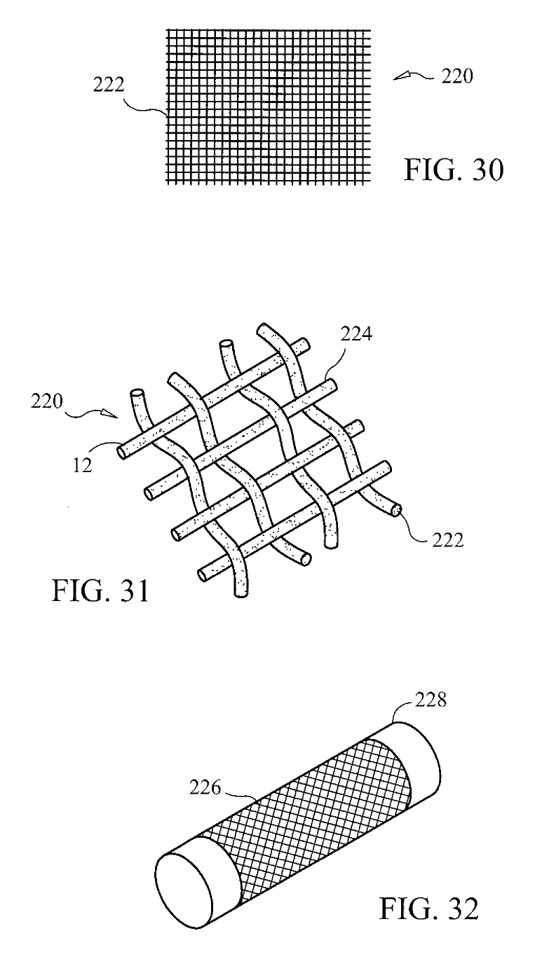

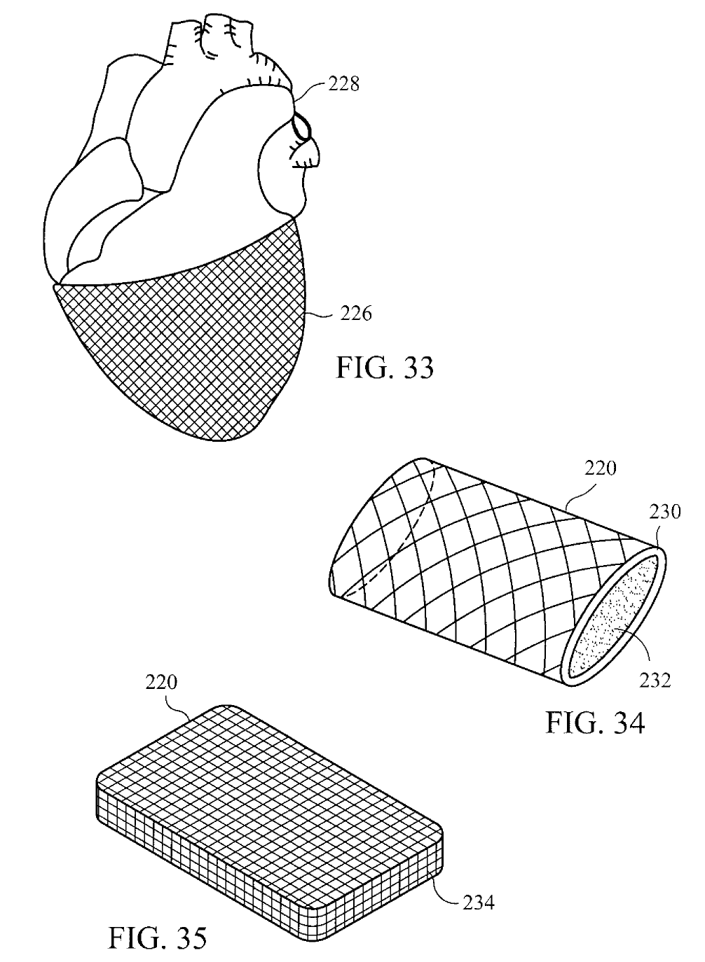

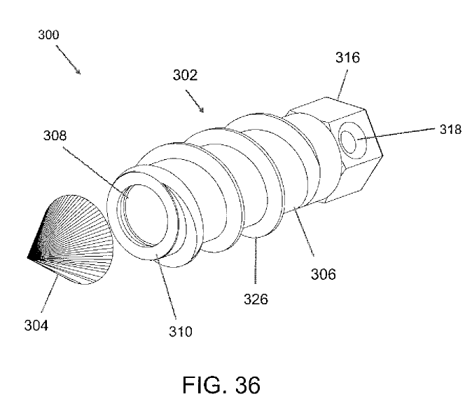

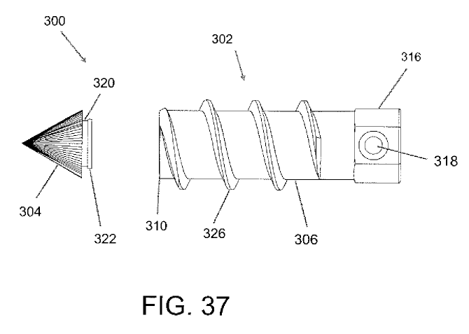

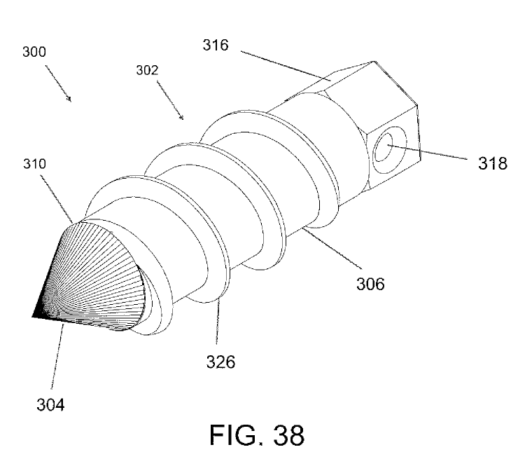

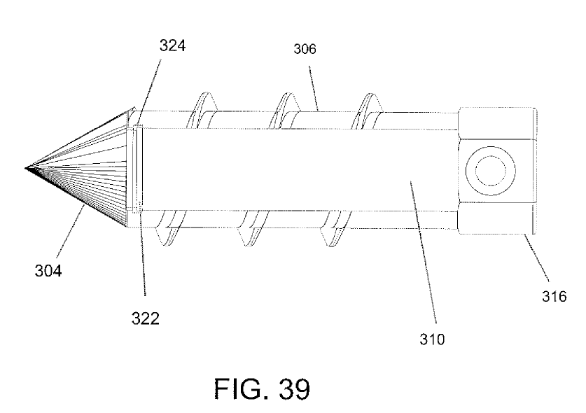

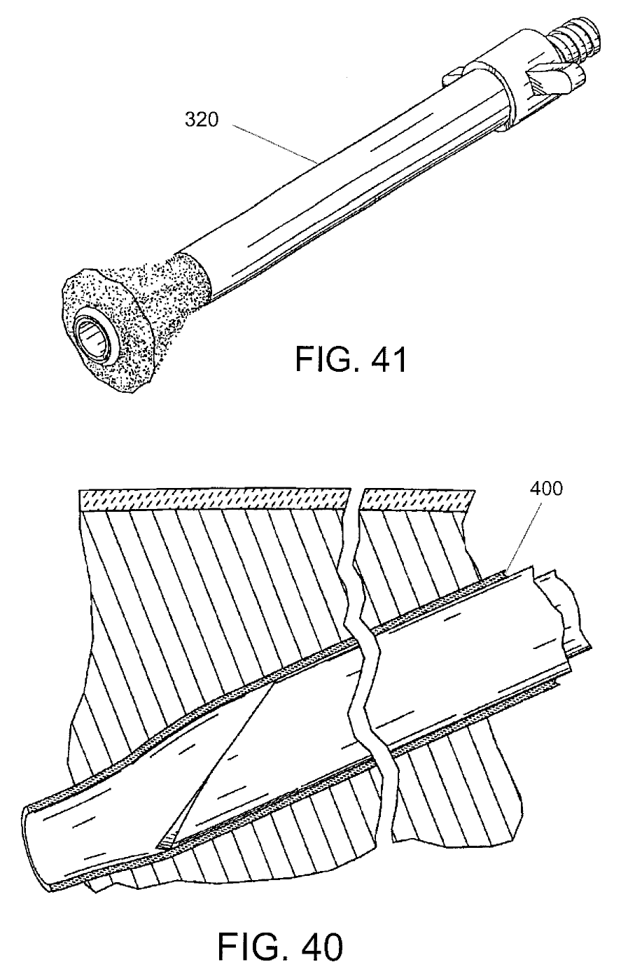

Referring to FIG. 7, the polymer coating may be made up of micro capsules 28, affixed to the medical implant 10. The pharmaceutical agent 12 is contained within the micro capsule 28. The micro capsules 28 may be bonded to the medical implant 10 with a biodegradable agent, such that as the biodegradable agent degrades, micro capsules 28 are released. Similarly, the micro capsules 28 may be made of a biodegradable material, such that as the micro capsules 28 degrade, the pharmaceutical agent 12 will be released.

Alternatively, the medical implant 10 may be made entirely of micro capsules 28 bonded together. The bonded micro-capsule 28 can be appropriately shaped and sized depending on the intended area of use. The micro capsules 28 may be bonded to together with a biodegradable agent, such that as the biodegradable agent degrades the micro capsules 28 are released. Similarly, the micro capsules 28 may be made of a biodegradable material, such that as the micro capsules 28 degrade the pharmaceutical agent 12 will be released.

Referring to FIG. 8A, the medical implant 10 may include reservoirs 30 therein for receiving and holding a pharmaceutical agent 12. The reservoirs openings may have uniform diameters or have different diameters. As shown in FIG. 5B, the openings in the reservoirs 30 may be covered with a polymer plug 31. The pharmaceutical agent 12 is released by seeping through the polymer plug 31. The release rate of the pharmaceutical agent 12 may be controlled by controlling the thickness of the polymer plug 31. Reservoirs 30 with thicker plugs 31 will release the pharmaceutical agent 12 at a slower rate than reservoirs 30 with a thinner plug 31. In an embodiment, the polymer plug 31 is a biodegradable plug. As the biodegradable plug degrades, the pharmaceutical agent 12 within the reservoir 30 is released.

Each of the reservoirs 30 may contain the same pharmaceutical agent 12, having the same dosage. Alternatively, the dosage of the pharmaceutical agent 12 in each of the reservoirs 30 may be different. Additionally, each of the reservoirs 30 may contain a different pharmaceutical agent 12 therein.

While in the foregoing FIGURES, the medical implant 10 was depicted in one embodiment as a stent, in other embodiments, similar techniques may be used to coat other types of implantable medical devices, such as hip and knee replacements (total and partial), spinal implants, scaffolds, biological implants or grafts, tissue grafts, screws, plates, rods, prosthetic devices, etc.

Additionally, a wide array of types of drugs may be delivered in a similar fashion as described above. For example, steroidal, nonsteroidal, pain relieving drugs, binary agents, hormones, cells, fetal cells, stem cells, bone morphogenic proteins (BMPs), enzymes, proteins, RNA, beneficial viruses and other agents may be delivered intraoperatively or postoperatively. In this regard, the coated medical implant 10 may advantageously be used as a multimodal treatment regimen with postoperative analgesic pain relief and accelerate tissue healing. This may be particularly advantageous for cementless implantation, disc replacement, tissue grafts, cellular therapy, gene therapy, implanted organs such as kidney transplants or partial implants, among other applications.

The medical implant 10 can be positioned in the body of the patient using known surgical techniques. For example the medical implant 10 can be positioned in the body of the patient using minimally invasive surgical techniques. In an exemplary embodiment. A balloon dissection device 402, as disclosed in U.S. Pat. No. 6,042,596, to Bonutti, the contents of which are incorporated by reference, and shown in FIG. 40, can be used to provide access and space for insertion of the medical device 10.

Referring to FIG. 9, an energy unit 32 may be used to control the release rate of the pharmaceutical agent 12 on the medical implant 10. The energy unit 32 provides an appropriate amount (e.g. frequency and amplitude) of energy signal 33 to the medical implant 110 which can be used to control the release rate of the pharmaceutical agent 12. For example, initially the pharmaceutical agent 12 is released by eluting through the polymer coating 22, where the release rate of the pharmaceutical agent 12 is a function of the properties of the polymer coating 22. The application of an energy signal 33 to the medical implant 12 changes the physical properties of the polymer coating 22, increasing the release, or providing a bolus or burst of, the pharmaceutical agent 12. The energy unit 32 may heat up the medical implant 10 increasing the release rate of the pharmaceutical agent 12. The energy unit 32 may be an intracorporeal or extracorporeal energy unit.

Additionally, the energy unit 32 may also heat up the treatment site, locally increasing vascularity at the treatment site. The localized increasing in temperature increases the permeability of the local tissue, allowing for an increased and more efficient adsorption of the pharmaceutical agent 12 into the treatment site. Furthermore, in response to localized increase in temperature, which can be perceived as physical damage or an infection to the local area, the local cells may release beneficial proteins, enzymes, hormones, etc.

Additionally, where the pharmaceutical agent 12 includes cells having a biologic agent therein, the energy unit 32 may be used to disrupt the cell walls to release the biologic agent. The cells are selected or designed to react to a given energy signal 33 to release the enclosed agent. The implant can include different cells which react to different energy signals 33 to release the enclosed biologic agents. The biologic agent can include genes, RNA, DNA, or viruses. The disruption of the cell wall causes the release of the biologic agent, which would then allow the biologic agent to differential on its own.

Referring to FIG. 10, an exemplary energy unit 32 is shown which utilizes acoustic waves to provide an energy signal to the medical implant 10 and pharmaceutical agent 12. The energy unit 32 includes an acoustic signal source 34 connected to a transmitter 36 through conductors 38 and 40. Transmitter 36 includes a piezoelectric transducer or any other acoustic source capable of emitting acoustic waves receivable by the implant 10. The frequency of the acoustic waves may be in any suitable range including, but not limited to, frequencies in the ultrasonic (frequencies generally higher than 20 KHz), sonar (generally 25-100 KHz), medical ultrasonic (generally 1-10 MHz), and microwave acoustic (frequencies generally over 50 MHz) ranges.

Although any appropriate energy unit can be used, another energy source that has been used extensively in medical applications is extracorporeal shockwaves (ESW). The ESW system includes an energy source (the shockwave generator), a focusing system, and a coupling mechanism.

The shockwave generator can take the form of electrohydraulic, piezoelectric, and electromagnetic energy. In an electrohydraulic generator, an electrical discharge of a high-voltage current occurs across a spark-gap electrode located within a fluid-filled container. The electric discharge results in a vaporization bubble, which expands and immediately collapses, thereby generating a high-energy pressure wave. In a piezoelectric generator, hundreds-to-thousands of ceramic or piezo crystals are set in a fluid-filled container and are stimulated with a high-energy electrical pulse. The high-energy electrical pulse vibrates or rapidly expands the crystals, leading to a shockwave that can be propagated through the fluid. In an electromagnetic generator, an electrical current is applied to an electromagnetic coil mounted within a fluid-filled cylinder. The magnetic field causes an adjacent metallic membrane to be repelled by the coil, resulting in extremely rapid movement of the membrane, therapy producing a shaped shockwave. Exemplary shockwave generators are provided in U.S. Pat. Nos. 2,559,227, 4,947,830 and 5,058,569, the contents of which are herein incorporated by reference.

The focusing system concentrates and directs the shockwave energy into the body of the patient. For example, an electrohydraulic system utilizes the principle of the ellipse to direct the energy created from the spark-gap electrode. Piezoelectric systems arrange their crystals within a hemispherical dish, arranged so that the energy produced is directed toward one focal point. Electromagnetic systems use either an acoustic lens or a cylindrical reflector to focus their waves.

The coupling system transmits the energy created by the shockwave generator to the skin surface and through body tissues into the patient. The coupling system can take the form of a large water bath in which the patient is submerged. Alternatively, the coupling system can be small pools of water or fluid-filled cushions with a silicone membrane to provide air-free contact with the patient's skin.

The above exemplary energy unit 32 may transmit a steady energy signal to the medical implant 10. It is also contemplated that the energy unit 32 may provide a pulsated energy signal to the medical implant 10, resulting in pulsated treatment to the treatment site. Alternatively, the frequency and/or amplitude of the energy signal may be modulated.

In addition to the energy unit 32 described above, the energy unit 32 of the present invention may optionally provide radio frequency (RF), magnetic, electro magnetic (EM), acoustic, microwave, laser, optical, thermal, vibratory, or extracorporeal shockwave (ESW) energies, alone or in any combination thereof to the medical implant 10. Furthermore, the frequency and/or amplitude of the transmitted energy signal may be adjusted, depending of the depth, size, density, location, etc. of the treatment site.

Referring to FIGS. 5A and 9, the energy unit 32 is used in conjunction with a medical implant 10 including a porous coating 24. The porous coating 24 acts as a membrane to diffuse the pharmaceutical agent 12. Initially, the pores 44 in the porous coating 24 are closed or significantly small to eliminate or severely restrict release of the pharmaceutical agent 12. In operation, the energy unit 32 may be positioned over the medical implant 10 and provide an energy signal to react with the porous coating 24, increasing the size of the pores 44, to thereby release the pharmaceutical agent 12. After a therapeutic amount of the pharmaceutical agent 12 has been released, the applied energy signal may be discontinued, closing the pores 44.

Referring to FIG. 5B, the pores 44 may include at least two different opening diameters 44a and 44b. The different diameter openings correspond to different size ranges of opening diameters. For example, the first opening diameter 44a corresponds to a first range of pore opening diameters and the second opening diameter 44b corresponds to a second range of pore opening diameters. The different opening diameter ranges 44a and 44b are attuned to react at different frequencies/wavelengths, allowing for the selective release of different pharmaceutical agents 12a and 12b therein. In operation, the energy unit 32 may be positioned over the medical implant 10, providing an energy signal at a first frequency/wavelength range to react with corresponding first diameter pores 44a, for example by increasing the size of the pores 44a, to selectively release the first pharmaceutical agent 12a therein. After a therapeutic amount of the first pharmaceutical agent 12a has been released, the signal may be discontinued.

Optionally, the energy unit 32 may provide an energy signal at a second frequency/wavelength range to react with corresponding second diameter pores 44b, for example by increasing the size of the second diameter pores 44b, to selectively release the second pharmaceutical agent 12b therein. After a therapeutic amount of the second pharmaceutical agent 12b has been released, the energy signal may be discontinued.

Referring to FIGS. 6 and 9, the energy unit 32 is used in conjunction with a medical implant 10 including a biodegradable coating 26. In operation, the energy unit 32 may be positioned over the medical implant 10, providing an energy signal at a frequency to react with the biodegradable coating 26, partially breaking-up or fragmenting the biodegradable coating 26 from the medical implant 10. The applied energy signal increases the degradation, fragmentation, or dissolution rate of the biodegradable coating 26. After the desired dissolution rate of the biodegradable coating 26 has been achieved, the energy signal may be discontinued. The increased dissolution rate of the biodegradable coating 26 accelerates the release of the pharmaceutical agent 12 therein. At set time intervals or as needed, the energy unit 32 may be used to selectively increase the dissolution rate of the biodegradable coating 26 to selectively increase the release of the pharmaceutical agent 12 therein.

In an alternate embodiment, the medical implant 10 may include a plurality of layers or sections of biodegradable coatings 26, each including a different therapeutic amount of a pharmaceutical agent 12. The energy unit 32 may be used to apply an energy signal to selectively release a layer of the biodegradable coating 26, releasing a corresponding therapeutic amount of a pharmaceutical agent 12. Each of the layers or sections of the biodegradable coating 26 may be released as needed or at set time intervals.

The biodegradable coating 26 may include polyactic acid (“PLA”), polyglycolic acid (“PGA”), and copolymers thereof. The degradation rate of the biodegradable coating can be controlled by the ratio of PLA to PGA, or by the thickness or density of the coating. Additionally, the biodegradable coating 26 may also include collagen, cellulose, fibrin, or other cellular based compounds. In the prior art, degradation had to be set prior to implantation by selecting the above-parameters based on the anticipated clinical situation. With the present invention, the degradation can be changed to adapt to the actual clinical situation.

In an exemplary delivery method, the pharmaceutical agent 12 is delivered from a polymer matrix. Solution of the pharmaceutical agent 12, prepared in a solvent miscible with the polymer carrier solution, is mixed with the solution of polymer at a final concentration range. Polymers are biocompatible (i.e., not elicit any negative tissue reaction or promote mural thrombus formation) and degradable, such as lactone-based polyesters or copolyesters, e.g., polylactide, polycaprolacton-glycolide, polyorthoesters, polyanhydrides; poly-aminoacids; polysaccharides; polyphosphazenes; poly(ether-ester) copolymers, e.g., PEO-PLLA, or blends thereof. Nonabsorbable biocompatible polymers are also suitable candidates. Polymers such as polydimethyl-siolxane; poly(ethylene-vingylacetate); acrylate based polymers or copolymers, e.g., poly(hydroxyethyl methylmethacrylate, polyvinyl pyrrolidinone; fluorinated polymers such as polytetrafluoroethylene; cellulose esters.

Polymer/agent mixture is applied to the surfaces of the medical implant by either dip-coating, or spray coating, or brush coating or dip/spin coating or combinations thereof, and the solvent allowed to evaporate to leave a film with entrapped pharmaceutical agent.

In an alternative exemplary delivery method, the pharmaceutical agent 12 is delivered through a polymer membrane coating. A medical implant 10 is dipped into a solution of the pharmaceutical agent 12 saturated in an organic solvent, such as acetone or methylene chloride. A solution of polymer is applied to the medical implant 10 as detailed above. This outer layer of polymer will act as diffusion-controller for release of drug.

Referring to FIGS. 7 and 9, the energy unit 32 is used in conjunction with a medical implant 10 including a micro capsule 28 coating. In operation, the energy unit 32 may be positioned over the medical implant 10, providing an energy signal to react with the micro capsules 28, breaking off a number of the micro capsules 28 from the medical implant 10. The applied energy signal increases the degradation, fragmentation, or dissolution rate of the micro capsules 28 to accelerate the release of the pharmaceutical agent 12. After a therapeutic amount of the pharmaceutical agent 12 has been released, the energy signal may be discontinued.

Referring to FIGS. 8A-B and 9, the energy unit 32 is used in conjunction with a medical implant 10 including plugged reservoirs 30. In operation, the energy unit 32 may be positioned over the medical implant 10, providing energy signal at a frequency to react with the biodegradable plugs, breaking off a number of the biodegradable plugs from the medical implant 10. The applied energy signal increases the degradation, fragmentation, or dissolution rate of the biodegradable plugs to accelerate the release of the pharmaceutical agent 12. After a therapeutic amount of the pharmaceutical agent 12 has been released, the energy signal may be discontinued.

The reservoirs 30 may have at least two different opening diameters, such that different diameter plugs are provided on the reservoir openings. The different opening diameters are attuned to react at different frequency/wavelength ranges, allowing for the selective release of pharmaceutical agents 12 therein.

In operation, the energy unit 32 may be positioned over the medical implant 10, providing energy signal at a first frequency/wavelength range, reacting with corresponding first diameter biodegradable plugs, rupturing, and/or breaking off a number of the biodegradable plugs from the medical implant 10 to selectively release a first pharmaceutical agent 12. The applied energy signal increases the degradation, fragmentation, or dissolution rate of the biodegradable plugs to accelerate the release of the first pharmaceutical agent 12. After a therapeutic amount of the first pharmaceutical agent 12 has been released, the energy signal may be discontinued.

Optionally, the energy unit 32 may provide an energy signal at a second frequency/wavelength range to react with corresponding second diameter biodegradable plugs, rupturing, and/or breaking off a number of the biodegradable plugs from the medical implant 10 to selectively release a second pharmaceutical agent 12. After a therapeutic amount of the second pharmaceutical agent 12 has been released, the energy signal may be discontinued.

Referring to FIGS. 9 and 11, the medical implant may be a biodegradable implant 46 impregnated with the pharmaceutical agent 12. The biodegradable implant 46 can be made of a biodegradable polymer, collagen, cellulose, fibrin, or other cellular based compounds. Similar to above (See FIG. 2), a pharmaceutical agent 12 may be affixed to the biodegradable implant 46 by coating, mixing, or bonding the pharmaceutical agent 12 to a polymer coating 22 applied to the biodegradable medical implant 10. In operation, the energy unit 32 may be positioned over the biodegradable implant 46, providing an energy signal at a frequency to react with the biodegradable implant 46, partially breaking-up or fragmenting a portion of the biodegradable implant 46. The applied energy signal increases the degradation, fragmentation, or dissolution rate of the biodegradable implant 46, to accelerate the release of the pharmaceutical agent 12. After a therapeutic amount of the pharmaceutical agent 12 has been released, the energy signal may be discontinued.

Referring to FIGS. 9 and 12, the biodegradable implant 46 may be made up of a plurality of layers or sections 48, each including a different therapeutic amount of a pharmaceutical agent 12. The energy unit 32 may be used to apply an energy signal to selectively release a layer 48 of the biodegradable implant 46, releasing the corresponding therapeutic amount of a pharmaceutical agent 12. Each of the layers or sections 48 of the biodegradable implant 46 may be released as needed or at set time intervals. The biodegradable implant 46 may be made of polyactic acid (“PLA”), polyglycolic acid (“PGA”), and copolymers thereof. The degradation rate of the biodegradable implant can be controlled by the ratio of PLA to PGA, or by the thickness or density of the coating. Additionally, the biodegradable implant 46 may be made or collagen, cellulose, fibrin, or other cellular based compounds.

In an embodiment, the biodegradable implant 46 is a biological implant, which can include bone, collagen, cartilage, muscle, tendon, ligaments, or other tissue graft material. The biologic implant can be formed by methods disclosed in U.S. Pat. No. 6,468,289, to Bonutti, and U.S. Pat. No. 6,776,938, to Bonutti, the contents of which are incorporated by reference.

In an alternate embodiment, the biodegradable implant 46 is made up of micro capsules 28. The pharmaceutical agent 12 is contained within the micro capsule 28. In operation, the energy unit 32 may be positioned over the biodegradable implant 46, providing an energy signal at a frequency to react with the biodegradable implant 46, breaking off a number of the micro capsules 28. The applied energy signal increases the degradation, fragmentation, or dissolution rate of the micro capsules 28 to accelerate the release of the pharmaceutical agent 12. After a therapeutic amount of the pharmaceutical agent 12 has been released, the energy signal may be discontinued.

The biodegradable implant 46 can be positioned in the body of the patient using known surgical techniques. For example, the biodegradable implant 46 can be positioned in the body of the patient using minimally invasive surgical techniques. In an exemplary embodiment, an expandable cannula 400, as shown in FIG. 40, can be used to provide access for insertion of the biodegradable implant 46. As previously described, a balloon dissection device can be used to provide access and space for insertion of the biodegradable implant 46.

The biodegradable implant 46 may also include an adhesive to bond the biodegradable implant 46 to the implantation site. Such adhesives may include cyanoacrylate adhesives, hydrogel adhesives, monomer and polymer adhesives, fibrin, polysaccharide, Indermil® or any other biocompatible adhesive. Alternatively, the biodegradable implant 46 may be intra corporeally welded to the treatment to the treatment site, using surgical welding techniques.

A biodegradable implant 46 filled with one or more therapeutic agents may form a drug cocktail implant. The therapeutic agents selected to be bonded with the biodegradable implant 46 may be specifically tailored to the needs of the patient. Once placed within the body, the therapeutic agent is slowly released to the surrounding tissue.

The present invention contemplates that energy unit 32 can be placed either extra or intra corporeally. Although energy unit 32 can be placed in vivo in any number of ways, it may be beneficial to use a percutaneous procedure. Referring to FIG. 13, an expandable cannula 50 may be used to position an energy unit 32 in proximity to the medical implant 10 of the present invention. Exemplary expandable cannulas are disclosed in U.S. Pat. No. 5,961,499, to Bonutti, and U.S. Pat. No. 6,749,620, to Dubrul et al., the contents of which are incorporated by reference. In one practical application of this embodiment, the medical implant 10 may be surgically positioned on or proximal to an artery, vein, or other vessel. The expandable cannula 50 is inserted through the skin 54 of the patient, until a tip portion 56 is proximal to the medical implant 10. The expandable cannula 50 is expanded, increasing the diameter of the expandable cannula 50. The energy unit 32 is positioned through the expandable cannula, in proximity to the medical implant 10. A power source 58 provides energy to the energy unit 32, such that an energy signal is transmitted to the medical implant 10, thereby releasing the pharmaceutical agent 12.

In the above embodiments, the present invention utilizes an external energy unit 32 or external power source 58 to provide an energy signal to the medical implant 10. Referring to FIG. 14 an internal energy unit 60, including an internal power supply 62, may be surgically or percutaneously positioned proximal to the medical implant 10. Imaging techniques, such as MRI, CT scan, ultrasound, x-ray, fluoroscope, etc., may be used to facilitate the implantation of the internal energy unit 60 and medical implant 10. Similar to FIG. 13, an expandable cannula 50 may be used to position an internal energy unit 60 in proximity the medical implant 10. The expandable cannula 50 is inserted through the skin 52 of the patient, until a tip portion 56 is proximal to the medical implant 10. The expandable cannula 50 is expanded, increasing the diameter of the expandable cannula 50. The internal energy unit 60 is positioned through the expandable cannula 50, in proximity to the medical implant 10. The expandable cannula 60 is removed, and the insertion site sealed.

The internal energy signal unit 60 includes a battery for providing power. The battery has a limited life span, upon the expiration of which the internal energy unit may be surgically or percutaneously removed and/or replaced.

Alternatively, the internal energy unit may include a rechargeable battery. Referring to FIG. 15, the rechargeable battery 64 may be recharged by positioning an external energy unit 32 on the skin of the patient's body, adjacent to and aligned with the rechargeable battery 64. An energy signal is transmitted through the body of the patient to the rechargeable battery 64. In one embodiment, the rechargeable battery 64 includes a piezoelectric device 66. An exemplary piezoelectric device 66 includes a ferromagnetic plate 68 attached to a ceramic disk 70. The energy signal from the external energy unit 32 causes the piezoelectric ceramic disk 70 to vibrate, thereby generating a voltage which recharges battery 64. An exemplary energy signal system for non-invasively recharging an implanted rechargeable battery is disclosed in U.S. Pat. No. 5,749,900, to Schroeppel, the contents of which are incorporated by reference. Alternatively, the external energy unit 32 may be percutaneously or transcutaneously positioned proximal to the rechargeable battery 64.

In the above embodiment, the rechargeable battery 64 is described as requiring an external energy unit 32 to be recharged. However it is contemplated, that the rechargeable battery 64 can include a self-recharging mechanism. The self-recharging mechanism utilizes the movement of the patient to create electricity to recharge the rechargeable battery 64.

Referring to FIG. 16, the internal energy unit 60 may include a control unit 72. In operation, the control unit 72 is configured to selectively activate the internal energy unit 60 at pre-programmed set time intervals.

Alternatively, the control unit 72 may be controlled from an external unit. The control unit 72 further includes a transceiver 74 configured to receive an external signal. The transceiver 74 activates or deactivates the internal energy unit 60 in response to an external signal. For example, the transceiver 74 may be configured to receive an RF signal.

Referring to FIGS. 9, 17, and 18, the energy unit 32 is used in conjunction with a medical implant 10 including a stable, non-degradable coating 80. The non-degradable coating 80 acts as a barrier to substantially prevent the release of the pharmaceutical agent 12. In operation, the energy unit 32 may be positioned over the medical implant 10 and provide an energy signal to react with the coating 80, resulting in the formation of cracks 82 in the non-degradable coating 80. The cracks 82 allow for the pharmaceutical agent 12 to be released from the medical implant 10.

The medical implant 10 and the non-degradable coating 80 have different rates of thermal expansion. For example, the medical implant 10 has a greater rate of thermal expansion than the non-degradable coating 80. As the energy unit 32 applies an energy signal, heating the medical implant 10 and the non-degradable coating 80, the medical implant 10 expands at a greater rate than the non-degradable coating 80. The differential rates of expansion of the medical implant 10 and the non-degradable coating 80 results in the formation of cracks in the non-degradable coating 80.

In another embodiment, the application of energy from the energy unit 32 changes the material properties of the non-degradable coating 80. For example, upon initial application to and insertion of the medical implant 10 the non-degradable coating 80 has elastic material properties. The elastic material properties allow the non-degradable coating 80 to expand, contract, and deform with the medical implant 10. As the energy unit 32 applies an energy signal to the medical implant 10 and the non-degradable coating 80, the material properties of the non-degradable coating 80 change, wherein the non-degradable coating 80 becomes increasingly brittle. With continued application of energy, the non-degradable coating 80 become sufficiently brittle such that cracks 82 are formed in the non-degradable coating 80 with the expansion, contraction, or deformation of the medical implant 10. Alternatively, a continued application of energy from the energy unit 32 may itself result in the formation of cracks 82 in the non-degradable coating 80.

Referring again to FIG. 17, the medical implant 10 includes a polymer coatings 84 interposed between the medical implant 10 and the non-degradable coating 80. The polymer coating 84 is impregnated with the pharmaceutical agent 12. Upon the formation of cracks 82 in the non-degradable coating 80, the pharmaceutical agent 12 elutes from the non-degradable coating 80. If the polymer coating 84 is biodegradable, such that upon the formation of cracks 82 in the non-degradable coating 80, the polymer coating 84 degrades releasing the pharmaceutical agent 12. Alternatively, the polymer coating 84, like polymer coating 80 is made of a non-degradable material such that upon the formation of cracks 82, pharmaceutical agent 12 is released by diffusing through coating 84.

It is contemplated that multiple polymer coating layers can be interposed between the medical implant 10 and the non-degradable coating 80, where a pharmaceutical agent 12 is disposed within each of the polymer coatings layer. Each of the layers may contain the same pharmaceutical agent 12, having the same dosage. Alternatively, the dosage of the pharmaceutical agent 12 in each of the multiple polymer coating layers may be different. Additionally, each of the polymer coating layers may contain a different pharmaceutical agent 12 therein.

As previously noted, the non-degradable coating 80 acts as a barrier to substantially prevent the release of the pharmaceutical agent 12. This allows the medical implant 10 to be positioned in the patient prior to the need of the pharmaceutical agent 12. Only when the pharmaceutical agent 12 is required is the energy signal applied to form cracks 82 in the non-degradable coating 80 to release the pharmaceutical agent 12.

Referring to FIG. 19, the medical implant 10 of the present invention may include an energy sink 88. The energy sink 88 may be incorporated into the medical implant 10 or be positioned separate from the medical implant 10. The energy sink 88 is used to control the elution rate of the pharmaceutical agent 12 through polymer coating 22. For example, the energy sink 88 may be a heat sink, wherein the heat sink 88 is charged by the energy unit 32. Initially, the elution rate of the pharmaceutical agent 12 is dependent on the polymer coating 22, where the pharmaceutical agent 12 elutes though the polymer coating at a substantially steady rate. To increase the elation rate of the pharmaceutical agent 12, the energy unit 32 is used to charge the beat sink 88. The heat sink 88 produces a local increase in temperature, including an increase in the temperature of the polymer coating 22. The increase in the temperature of polymer coating 22 increases the elution rate of the pharmaceutical agent 12 through the polymer coating 22. Alternatively, the increase in temperature can increase the degradation rate of a degradable polymer coating 22, increasing the release of the pharmaceutical agent 12.

Additionally, the localized increase in temperature created by the heat sink 88 has beneficial effects, which include (but are not limited to): aiding in the alleviation of localized pain, fighting of local infections, and increasing vascular flow and permeability of vessels at the treatment site to control delivery of pharmaceutical agent 12. For example, a localized increasing in temperature increases the permeability of the local tissue, allowing for an increased and more efficient absorption of the pharmaceutical agent 12 into the treatment site.

In an alternative embodiment, the energy sink 88 is a pH sink, wherein the pH sink 88 may be incorporated into the medical implant 10 or be positioned separate from the medical implant 10. The pH sink 88 is configured to absorb energy from the energy unit 32, releasing a chemical to either increase or decreasing the local pH. The change in local pH can either increase or decrease the degradation rate of a degradable polymer coating 22, which in turn can control the release rate of a pharmaceutical agent 12. The pH sink 88 can be formed from calcium carbonate.

Additionally, the localized change in pH created by the pH sink 88 has beneficial effects, which include (but are not limited to): aiding in the alleviation of localized pain, fighting of local infections, and increasing vascular flow and permeability of vessels at the treatment site to control delivery of pharmaceutical agent 12. For example, a localized increase in pH increases the permeability of the local tissue, allowing for an increased and more efficient absorption of the pharmaceutical agent 12 into the treatment site.

The energy sink 88 may also be used to induce the release of beneficial enzymes, proteins, hormones, etc. from the cells in the treatment site. A localized increase in acidity and/or temperature can be perceived as a physical damage or an infection to the local area. In response, the local cells may release beneficial proteins, enzymes, hormones, etc.

In addition to the energy sinks 88 described above, the energy sink 88 of the present invention may optionally provide, magnetic, radiation, chemical, or thermal energies, alone or in any combination thereof, to the medical implant 10.

Referring to FIG. 20, the medical implant 10 is magnetically or electrically charged. Likewise, the pharmaceutical agent 12 is magnetically or electrically charged, such that the pharmaceutical agent 12 is magnetically or electrically bonded to the medical implant 10. The pharmaceutical agent is released as the bond between the medical implant 10 and the pharmaceutical agent 12 decreases. The magnetic or electrical bond between the medical implant 10 and the pharmaceutical agent 12 can gradually decrease over time, providing a controlled gradual release of the pharmaceutical agent 12. Alternatively, an external energy can be applied to increase the degradation of the bond between the medical implant 10 and the pharmaceutical agent 12, to provide an increased release rate of the pharmaceutical agent 12.

Upon depletion of the pharmaceutical agent 12 from the medical implant 10, the magnetic or electric charge permits additional pharmaceutical agents 12 to be bonded to the medical implant 10. Initially, the magnetic or electric charge on the medical implant can be recharged using an external energy unit. For example, an MRI device can be used to increase the magnetic charge of the medical implant 12. Charged pharmaceutical agents 12 can be injected into the patient, through the blood stream or adjacent to the medical implant 10, where the charges of the medical implant 10 and pharmaceutical agents 12 result in the bonding of the pharmaceutical agent 12 to the medical implant 10. This enables the medical implant 10 to be refilled with pharmaceutical agent 12, without removing the medical implant 10 from the body of the patient.

In an alternative embodiment, the medical system provides a medical implant having fillable reservoirs thereon. The reservoirs are filled with a pharmaceutical agent just prior to insertion into the body of the patient. This allows the medical implant to be specifically tailored for the patient. Referring to FIG. 21, the medical implant 90 includes a first body portion 92 having one or more reservoirs 94 therein. A pharmaceutical agent 12 is disposed within each of the reservoirs 94, wherein each reservoir 94 may contain the same pharmaceutical agent 12, having the same dosage. Alternatively, the dosage of the pharmaceutical agent 12 (and/or the composition of the agents) in each reservoir 94 may be different.

A cover portion 96 is placed on the first body portion 92, covering and sealing the pharmaceutical agent 12 within the reservoirs 94. The pharmaceutical agent 12 is released by eluting through the cover portion 96 and first body portion 92, wherein the elution rate is dependent of the thickness of the cover portion 96 and first body portion 92. For example, the cover portion 96 can have a uniform thickness allowing for a uniform elution rate therethrough. Alternatively, the cover portion can have a variable thickness, allow for a varying elution rate. Referring to FIG. 22, the thickness of the cover portion 96 increases across the medical implant 92, wherein each of the reservoirs 94a-c is covered by an increasingly thick cover portion 96. The elution rates of the pharmaceutical agent 12 in each of the reservoirs 94a-c decreases as the thickness of the cover portion 96 increases.

The cover portion 96 and/or first body portion 92 may be made of a bioerodible, bioabsorbable, material. The pharmaceutical agent 12 is released as the cover portion 96 and first body portion 92 degrade. The cover portion 96 and first body portion 92 can have a uniform degradation rate, allowing for uniform release rate of the pharmaceutical agent 12. Alternatively, the cover portion 96 and first body portion 92 can have a variable degradation rate, allow for a varying rate of release of the pharmaceutical agent 12. The biodegradable cover portion 96 and first body portion 92 may include resorbable polymers, such as polyactic acid (“PLA”), polyglycolic acid (“PGA”), and copolymers thereof. The degradation rate of the biodegradable cover portion 96 can be controlled by the ratio of PLA to PGA, or by the thickness or density of the coating. Additionally, the biodegradable cover portion 96 and first body portion 92 may also include collagen, cellulose, fibrin, or other cellular based compounds.

The cover portion 96 may be bonded to the first body portion 92, covering and sealing the pharmaceutical agent within the reservoirs 94, with an adhesive material. The adhesive material is a biocompatible adhesive.

Alternatively, ultrasonic vibratory energy is utilized to bond the cover portion 96 to the first body portion 92, covering and sealing the pharmaceutical agent within the reservoirs 94. The ultrasonic vibratory energy is at a frequency above that which can normally be detected by the human ear, that is, above 16 to 20 kilohertz. Although there are a wide range of frequencies which may be utilized, it is believed that it will be desirable to use ultrasonic energy having a frequency of between 20 kilohertz and 70 kilohertz. However, higher frequency vibratory energy could be utilized if desired.

The ultrasonic vibratory energy may be continuously applied, pulsed or modulated in various fashions. Any one of many known transducers may be utilized to change electrical energy into mechanical vibrations having an ultrasonic frequency. The transducers may be piezoelectric, ferroelectric, or magnetostrictive. One commercial source of apparatus which may be utilized to provide ultrasonic vibratory energy is Dukane Corporation, Ultrasonics Division, 2900 Dukane Drive, St. Charles, Ill. Of course, there are other sources of apparatus which can be utilized to provide ultrasonic vibratory energy.

The ultrasonic vibratory energy creates frictional heat at the areas where the cover portion 96 and the first body portion 92 are disposed in engagement with each other. The frictional heat provided by the ultrasonic vibratory energy is effective to heat the material of the cover portion 96 and the first body portion 92 into its transition temperature range.

Once the materials of the cover portion 96 and the first body portion 92 have been heated into its transition temperature range by the ultrasonic vibratory energy, the plastic material of the cover portion 96 and the first body portion 92 loses its rigidity and becomes soft and viscous. The softened material of the cover portion 96 and the first body portion 92 are moldable and flow, when subjected to pressure, together bonding the cover portion 96 and the first body portion 92.

Although generally described as using ultrasonic energy, it is again understood that other types of energy or combination of energies can be utilized to provide heat energy. These types of energy or combination of energies can include, but not be limited to, radio frequency (RF) energy, laser energy, microwave energy, ultrasound energy, and contact heating energy.

In one application, the medical practitioner selects a medical implant 10 having the appropriate number of reservoirs 94. Pharmaceutical agents 12 are placed in the reservoirs 94. Each of the reservoirs 94 may contain the same pharmaceutical agent 12, having the same dosage. Alternatively, the dosage of the pharmaceutical agent 12 (and/or the composition of the agents) in each of the reservoirs 94 may be different. A cover portion 96 is selected depending on the desired elution rate. A uniformly thick cover portion 96 is selected for a uniform elution rate or a varying thickness cover portion 96 is selection for a non-uniform elution rate. The cover portion 96 is bonded to the first body portion 92, covering the reservoirs. The medical implant is positioned in the body of the patient at the treatment site.

One potential advantage of this embodiment is that it allows the practitioner to adopt the pharmaceutical agent(s) and/or release characteristics of the medical implant 90 to a given clinical situation. For example, if an intra-operative biopsy reveals a certain pathology, a cocktail of pharmaceutical agents 12 specifically tailored for this pathology can be placed in the reservoirs 94. Additionally, the release of these pharmaceutical agents 12 can be controlled by the selection of first body portion 92 and cover portion 96. The present invention also contemplates the use of energy to control the release after implantation. Although reservoirs 94 are shown in a generic implant 90, this embodiment can be applied to any specific implant type.

Referring to FIG. 23, an example of a medical implant 100 utilized to fasten tissue portions is shown. It is contemplated that the medical implant 100 may be utilized to secure body tissue in many different ways. For example, the medical implant 100 may be utilized to secure one piece of body tissue to another piece of body tissue. The medical implant 100 may be utilized to secure soft tissue to soft tissue. It can also be used to secure soft body tissue to hard body tissue (bone). The medical implant 100 may be utilized to connect hard body tissue to hard body tissue in the manner disclosed in U.S. Pat. No. 6,238,395.

The medical implant 100 includes lower and upper sections 104 and 106. The lower section 104 has first and second recesses 108 and 110. As shown, the recesses 108 and 110 have the same configuration and are disposed the same distance from a central axis of the lower section 104. The illustrated recesses have elongated configurations with parallel longitudinal central axes which extend perpendicular to the central axis of the lower section 104. However, the recesses 108 and 10 could have many different configurations.

The upper section 106 includes first and second projections 114 and 116 extending therefrom. The first and second projections 114 and 116 have the same cross sectional configuration which corresponds to the cross sectional configuration of the recesses 108 and 110. The projections 114 and 116 have an elongated configuration with parallel longitudinal central axes which extend perpendicular to the central axis of the body 112 of the upper section 106. The projections 114 and 116 are disposed the same distance from a central axis of the upper section 106. It is contemplated that the projections 114 and 116 could have a configuration which is different than the above-described configuration.

A center projection 118 is disposed on the lower section 104 of the medical implant 100 at a location midway between the recesses 108 and 110. The projections 114 and 116 on the upper section 106 are received in the recesses 108 and 110 in the lower section 104 of the medical implant 100. This results in the upper section 106 of the medical implant 100 being positioned in a coaxial relationship with the lower section 104 of the medical implant 100. The center projection 118 is disposed midway between the projections 114 and 116 when they engage the recesses 108 and 110. The recesses 108 and 110 cooperate with the projections 114 and 116 to orient the upper section 106 of the medical implant 100 with the longitudinal axes of the projections 114 and 116 extending parallel to the longitudinal axis of the center section 118. Additional exemplary medical implant designs are also provided in U.S. patent application Ser. No. 10/779,978, the contents of which are herein incorporated by reference.

The lower and upper sections 104 and 106 may be bonded together covering and sealing the pharmaceutical agent 12 within the reservoirs 120. As previously discussed, an adhesive and/or thermal energy can be used in this regard.

The upper section 106 of the medical implant 100 includes a plurality of reservoirs 120 therein. A pharmaceutical agent 12 is disposed within each of the reservoirs 120, wherein each of the reservoirs 120 may contain the same pharmaceutical agents 12, having the same dosage. Alternatively, the dosage of the pharmaceutical agent 12 (and/or the composition of the agents) in each of the reservoirs 120 may be different.

A cover portion 122 is bonded onto the upper section 106, covering and sealing the pharmaceutical agents 12 within the reservoirs 120. The cover portion 122 may be bonded to the upper section 106 as described above. The pharmaceutical agent 12 is released by eluting through the upper section 106 and cover portion 122, wherein the elution rate is dependent of the thickness of the upper section 106 and cover portion 122. For example, the cover portion 122 can have a uniform thickness allow for uniform elution rate. Alternatively and as previously discussed, the cover portion can have a variable thickness, allow for a varying elution rate.

The upper section 106 and cover portion 122 may be made of a biocrodible, bioabsorbable, material. The pharmaceutical agent 12 is released as the tipper section 106 and cover portion 122 degrade. The upper section 106 and cover portion 122 can have a uniform degradation rate, allowing for uniform release rate of the pharmaceutical agent 12. Alternatively, the upper section 106 and cover portion 122 can have a variable degradation rate, allow for a varying rate of release of the pharmaceutical agent 12. The biodegradable upper section 106 and cover portion 122 may include resorbable polymers, such as polyactic acid (“PLA”), polyglycolic acid (“PGA”), and copolymers thereof. The degradation rate of the biodegradable upper section 106 and cover portion 122 can be controlled by the ratio of PLA to PGA, or by the thickness or density of the coating. Additionally, the biodegradable upper section 106 and cover portion 122 may also include collagen, cellulose, fibrin, or other cellular based compounds.

Referring to FIG. 24, in an embodiment, the medical implant 130 includes first and second sections 132 and 134 formed separately from each other. The first section 132 includes a top surface 136 having a closed wall portion 138 extending therefrom and defining a cavity 140 therein. A pharmaceutical agent 12 may be disposed within the cavity 140. The pharmaceutical agent 12 is disposed in the cavity 140 just prior to insertion into the body of the patient. This allows the medical implant to be specifically tailored for the patient.

The second section 134 is a cap having an aperture wall 141 configured to be fitted over and about the closed wall portion 138 of the first section 132. The second section 134 covers the cavity 140, sealing in the pharmaceutical agent 12. The second section 134 may be bonded to the first section 132 utilizing an adhesive material or and external energy source as described above.

The pharmaceutical agent 12 is released by eluting through the first and second sections 132 and 134, wherein the elution rate is dependent of the thickness of the first and second sections 132 and 134. For example, the first and second sections 132 and 134 can have a uniform thickness allowing for uniform elution rate. Alternatively, the first and second sections 132 and 134 can have a variable thickness, allowing for a varying elution rate.

Alternatively, the first and second sections 132 and 134 may be made of a degradable material. The pharmaceutical agent 12 is released as the first and second sections 132 and 134 degrade. The first and second sections 132 and 134 can have uniform degradation rates, allowing for uniform release of the pharmaceutical agent 12. Similarly, the first and second sections 132 and 134 can have a variable degradation rate, allowing for a varying rate of release of the pharmaceutical agent 12.

The biodegradable first and second sections 132 and 134 may include resorbable polymer such as polyactic acid (“PLA”), polyglycolic acid (“PGA”), and copolymers thereof. The degradation rate of the biodegradable first and second sections 132 and 134 can be controlled by the ratio of PLA to PGA, or by the thickness or density of the coating. Additionally, the biodegradable first and second sections 132 and 134 may also include collagen, cellulose, fibrin, or other cellular based compounds.

Referring to FIG. 25, in an embodiment, the closed wall portion 138 of the first section includes a divider member 142, bisecting the cavity 140 into first and second cavities 144 and 146. A pharmaceutical agent 12 may be disposed within each of the cavities 144 and 146, wherein each cavity 144 and 146 may contain the same pharmaceutical agents 12, having the same dosage. Alternatively, the dosage of the pharmaceutical agent 12 (and/or the composition of the agents) in each cavity 144 and 146 may be different.

It is further contemplated the cavity 140 can be subdivided into a plurality of cavities, wherein a pharmaceutical agent 12 may be disposed within each of the cavities. Each of the cavities may contain the same pharmaceutical agents 12, having the same dosage. Alternatively, the dosage of the pharmaceutical agent 12 (and/or the composition of the agents) in each of the cavities may be different. In this regard, the embodiment of FIG. 25 can be used when it is desirable to segregate two or more agents until implantation. Furthermore, if divider member 142 is not resorbable and does not allow diffusion therethrough, the agents will be kept separate even after implantation. This may be useful in situations in which both agents are needed, but cannot be given in a combined formulation.

Referring to FIG. 26, the medical implant 150 includes first and second sections 152 and 154 formed separately from each other. The first section 152 includes a top surface 156 having an inner closed wall portion 158 extending therefrom and defining an inner cavity 160 therein and an outer closed wall portion 162 surrounding the inner closed wall portion 158. An outer cavity 164 is defined between the inner and outer closed wall portions 158 and 162. A pharmaceutical agent 12 may be disposed in the inner cavity 160. The pharmaceutical agent 12 is disposed with the inner cavity 160 just prior to insertion into the body of the patient. This allows the medical implant to be specifically tailored for the patient.

The second section 154 is a cap having an aperture wall 166 configured to be fitted over and about the closed wall portion 158, wherein the aperture wall 166 is fitted into the outer cavity 164. The second section 154 covers the inner cavity 160, sealing in the pharmaceutical agent 12. The second section 154 may be bonded to the first section 152 utilizing an adhesive material or and external energy source as described above.

The pharmaceutical agent 12 is released by eluting through the first and second sections 152 and 154, wherein the elution rate is dependent of the thickness of the first and second sections 152 and 154. For example, the first and second sections 152 and 154 can have a uniform thickness, allowing for uniform elution rate. Alternatively, the first and second sections 152 and 154 can have a variable thickness, allowing for a varying elution rate.

Alternatively, the first and second sections 152 and 154 may be made of a degradable material. The pharmaceutical agent 12 is released as the first and second sections 152 and 154 degrade. The first and second sections 152 and 154 can have uniform degradation rates, allowing for uniform release rate of the pharmaceutical agent 12. Similarly, the first and second sections 152 and 154 can have a variable degradation rate, allow for a varying rate of release of the pharmaceutical agent 12.

The biodegradable first and second sections 152 and 154 may include resorbable polymers, such as polyactic acid (“PLA”, polyglycolic acid (“PGA”), and copolymers thereof. The degradation rate of the biodegradable first and second sections 152 and 154 can be controlled by the ratio of PLA to PGA, or by the thickness or density of the coating. Additionally, the biodegradable first and second sections 152 and 154 may also include collagen, cellulose, fibrin, or other cellular based compounds.

In a further embodiment, the first section 152 may include a lumen 168 in fluid communication with the inner cavity 160. The lumen 168 can serve as a drain, permitting the release of the pharmaceutical agent 12 therethrough. The lumen 168 can also serve as a mechanism in which cavity 160 can be filled (or refilled) with the desired pharmaceutical agents. In this regard, a one-way valve can be placed on lumen 168.

Referring to FIG. 27, the inner closed wall portion 158 of the first section 152 includes a divider member 170, bisecting the inner cavity 160 into first and second inner cavities 172 and 174. A pharmaceutical agent 12 may be disposed within each of the inner cavities 172 and 174, wherein each inner cavity 172 and 174 may contain the same pharmaceutical agent 12, having the same dosage. Alternatively, the dosage of the pharmaceutical agent 12 (and/or the composition of the agents) in each cavity 172 and 174 may be different.

It is further contemplated the inner cavity 160 may be further subdivided into a plurality of cavities, wherein a pharmaceutical agent 12 may be disposed within each of the cavities. Each of the cavities may contain the same pharmaceutical agents 12, having the same dosage. Alternatively, the dosage of the pharmaceutical agent 12 (and/or the composition of the agents) in each of the cavities may be different. In this regard, the embodiment of FIG. 27 can be used when it is desirable to segregate two or more agents until implantation. Furthermore, if divider member 170 is not resorbable and does not allow diffusion therethrough, the agents will be kept separate even after implantation. This may be useful in situations in which both agents are needed, but cannot be given in a combined formulation. For example, morphine and potassium cannot be given in a single solution so that one cavity can contain a morphine solution while the other can contain a potassium solution.

In further embodiment, the first section 152 may include first and second lumens 176 and 178 in fluid communication with the inner first and second cavities 172 and 174 respectively. As previously discussed, the lumens 176 and 178 can serve as drains and/or filling portals.

As disclosed in FIGS. 21-27 the pharmaceutical agent 12 is deposited directly into the reservoir or cavity of the medical implant. Referring to FIG. 28, the medical implant 190 includes a reservoir 192, configured for receiving a pharmaceutical agent 12. The reservoir 192 further includes an absorbent substrate material 194 positioned therein. The substrate material 194 is configured to receive the pharmaceutical agent 12, providing a stable medium for the pharmaceutical agent 12. The substrate material 12 is a nonbinding material, allowing the pharmaceutical agent 12 to be released through the medical implant 190. The absorbent material can be an mesh substrate or sponge made from polymer, polymer mixtures, copolymers, extracellular matrix components, proteins, collagen, fibrin or other bioactive agent, bone, or mixtures thereof.

Referring to FIG. 29, a suture 198 is used in conjunction with a medical implant 200 of the present invention to fasten tissue portions together. The medical implant 200 is used in a sterile, operating room environment to secure upper and lower layers of soft, human body tissue in linear apposition with each other. Thus, the two layers of human body tissue are approximated and held against movement relative to each other by the suture 208.

It is also contemplated that the suture 198 could extend through the medical implant 200 and/or be connected with body tissue in a manner similar to that disclosed in U.S. Pat. Nos. 5,584,862; 5,549,631; and/or 5,527,343. Of course, the suture 198 could be connected with body tissue in a different manner if desired.

Although the suture 198 could extend straight through the medical implant 200, in the illustrated embodiment of the invention, the suture 198 is wrapped around the closed wall portions 204 of the first section 202 of the medical implant 200.

The second section 206 is a cap having an aperture wall 208 configured to be fitted over and about the closed wall portion 204 of the first section 202. The second section 206 covers the cavity 210, sealing in the pharmaceutical agent 12. The second section 206 may be bonded to the first section 202 and the suture 198 utilizing an adhesive material or and external energy source as described above.

If an energy source is used, the source creates heat at the areas where the first section 202, second section 206, and the suture 198 are disposed in engagement with each other. The heat provided is effective to heat the material of the medical implant 200 into its transition temperature range while the material of the suture 198 remains at a temperature close to or below its transition temperature range. For example, the suture 198 may be formed of a material having a transition temperature range which is above 190 degrees Celsius. The suture retainer 198 may have a transition temperature range which, for the most part, is at a temperature below 190 degrees Celsius.

However, it should be understood that at least a portion or even the entire transition temperature range for the suture 198 could be co-extensive with the transition range for the medical implant 200. In fact, the transition temperature range of the suture 198 could extend below the transition temperature range of the medical implant 200. However, it is believed that it may be preferred to have the transition temperature range for the suture 198 above at least a portion of the transition temperature range of the medical implant 200.

Once the material of the suture retainer 198 has been heated into its transition temperature range, the plastic material of the suture retainer 198 loses its rigidity and becomes soft and viscous. The softened material of the medical implant 200 is moldable and flows, when subjected to pressure, around the suture 198 without significant deformation of the suture 198. However, the temperature range into which the suture 198 is heated and the pressure applied against the suture may result in some deformation of the suture 198.

Although it is contemplated that the suture 198 could be made of many different materials, the suture 198 may be formed of a plastic material which is a biopolymer. For example, the suture 198 may be formed of polyglycolide which is commercial available under the trademark “DEXON”. Polyglycolide is a crystalline material that melts at about 225° Celsius. However, the suture could be formed of a glycolide-based copolymer which is commercially available under the trademark “VICRYL”.

Exemplary methods of using medical implant of the present invention are provided in U.S. patent application Ser. No. 10/779,978, the contents of which are herein incorporated by reference.

Referring to FIG. 30 the medical implant of the present invention is a made of a mesh material 220. The mesh material 220 includes a plurality of interwoven interlaced, braided, or knitted fibers or filaments 222, wherein the fibers 222 can be directionally or non-directionally oriented. For example, the mesh material can be formed of orthogonal interwoven fibers 222.

The fibers 222 may be made of biocompatible and/or bioabsorbable material. For example, the fibers 222 may be formed from material which is polymeric, composite, metallic, ceramic, or combinations thereof. Furthermore, the fibers 222 may be made of or include body tissue including bone, collagen, cartilage, muscle, tendon, ligaments, or other tissue graft material.

Referring to FIG. 31, the mesh material 220 may be coated with a pharmaceutical agent 12. The pharmaceutical agent 12 being bonded to the surface of the fibers 222 by, for example, but not limited to, covalent bonding, ionic bonding, VanderWal forces, magnetic, etc. A primer layer can be placed on the fibers 222 and would be positioned between the fibers 222 and the agent 12. A top coat could be placed over the agent 12. The mesh material 220 may include a single layer or combination of pharmaceutical agents 12.

Alternatively, the mesh material 220 may include multiple layers of a single or a combination of pharmaceutical agents 12, which are coated onto the fibers 222 as previously described. The pharmaceutical agent 12 may be, for example, a drug, therapeutic agent, biological agent, or binary agent.

The pharmaceutical agent 12 may be affixed to the mesh material 220 by bonding the pharmaceutical agent 12 to the fibers 222 and coating the fibers 222 and pharmaceutical agent 12 with a polymer coat 224. The pharmaceutical agent 12 is released to the local treatment area by seeping through the polymer coating 224. The release rate of the pharmaceutical agent 12 is proportional to the thickness and/or permeability of the polymer coating 224.

Additionally, polymer coating 224 can be a degradable coating. The pharmaceutical agent 12 is initially released to the local treatment area by seeping through the polymer coating 224. As the polymer coating 224 degrades, the release rate of the pharmaceutical agent 12 may be increased.

In an embodiment, the mesh material 220 may include a gelatin substrate impregnated with the pharmaceutical agent 12. For example, the mesh material 220 is coated with the impregnated gelatin substrate and further coated with the polymer coat 224. The polymer coating 224 protects the integrity of the gelatin substrate, substantially preventing the release of the pharmaceutical agent 12. As the polymer coating 224 degrades, the gelatin substrate is at least partially exposed to body fluids, releasing the pharmaceutical agent 12. The gelatin substrate may be beneficial in storing active biologic agents, such as fetal cells, stem cells, viruses, RNA, etc. Although any suitable matrix can be used, a gelatin substrate is believed to be particularly useful for certain agents. Upon the degradation of the polymer coating 224, the biologic agents seep from the gelatin substrate.

The polymer coating 224 can include, for example, polyurethanes, polyethylene terephthalate (PET), PLLA-poly-glycolic acid (PGA) copolymer (PLGA), polycaprolactone (PCL) poly-(hydroxybutyrate/hydroxyvalerate) copolymer (PHBV), poly(vinylpyrrolidone) (PVP), polytetrafluoroethylene (PTFE, Teflon®, poly(2-hydroxyethylmethacrylate) (poly-HEMA), poly(etherurethane urea), silicones, acrylics, epoxides, polyesters, urethanes, parlenes, polyphosphazene polymers, fluoropolymers, polyamides, polyolefins, and mixtures thereof.

The pharmaceutical agent 12 can also be bonded to the mesh material 220 using other methods as previously described.

Referring to FIGS. 32 and 33, the mesh material 220 forms a mesh band 226 for positioning about an organ 228 such as intestine, vessel or a heart of a patient. The mesh band 226 may be positioned about the organ 228 to provide support to, aid in the function of, or healing of the organ 228. The pharmaceutical agent 12 is then released to the organ 228 and the surrounding area. The release rate of the pharmaceutical agent 12 can be controlled as previously described. Furthermore, it is contemplated that the present invention may be used with bariatric surgery, colorectal surgery, plastic surgery, gastroesophageal reflex disease (GERD) surgery, or for repairing hernias

Referring also to FIG. 9, an energy unit 32 may be used to control the release rate of the pharmaceutical agent 12 from the mesh material 220. The energy unit 32 provides an appropriate amount (e.g. frequency and amplitude) of energy signal 33 to the mesh material 220 which can be used to control the release rate of the pharmaceutical agent 12. For example, initially the pharmaceutical agent 12 is released by eluting through a polymer coating 224, where the release rate of the pharmaceutical agent 12 is a function of the properties of the polymer coating 224. The application of an energy signal 33 to the mesh material 220 changes the physical properties of the polymer coating 224, increasing the release, or providing a bolus or burst of, the pharmaceutical agent 12.

The energy unit 32 may heat up the mesh material 220, increasing the release rate of the pharmaceutical agent 12. Additionally, the energy unit 32 may also heat up the treatment site, locally increasing vascularity at the treatment and increasing absorption of the pharmaceutical agents 12. The energy unit 32 may be an intracorporeal or extracorporeal energy unit.

The mesh material 220 may include an energy sink. The energy sink may be incorporated into the mesh material or be positioned separate from the mesh material 220. For example, at least some of the fibers 222 can be electric or thermal conductive fibers or have electric or thermally conduct particles, such as iron, incorporated therein or thereon.

The energy sink is used to control the elution rate of the pharmaceutical agent 12 through mesh material 220. For example, the energy sink may be a heat sink, wherein the heat sink is charged by the energy unit 32. Initially, the elution rate of the pharmaceutical agent 12 is dependent on a polymer coating 224, where the pharmaceutical agent 12 elutes though the polymer coating 224 at a substantially steady rate. To increase the elution rate of the pharmaceutical agent 12, the energy unit 32 is used to charge the heat sink. The heat sink produces a local increase in temperature, including an increase in the temperature of the polymer coating 224. The increase in the temperature of polymer coating 224 increases the elution rate of the pharmaceutical agent 12 through the polymer coating 224. Alternatively, the increase in temperature can increase the degradation rate of a degradable polymer coating, increasing the release of the pharmaceutical agent 12.

Additionally, the localized increase in temperature created by the heat sink has beneficial effects, which include (but are not limited to): aiding in the alleviation of localized pain, fighting of local infections, and increasing vascular flow and permeability of vessels at the treatment site to control delivery of pharmaceutical agent 12.

Referring to FIG. 34, the mesh material 220 may be used to form a mesh pouch 230 for implantation to a treatment site in the body of the patient. The mesh pouch 230 may be implanted in the body of the patient using minimally invasive surgical techniques, such as using an expandable cannula or balloon dissection device. The mesh pouch 230 may include or may be filled an pharmaceutical agent 232, such as a therapeutic substances or drugs, like antibiotics, hydroxypatite, anti-inflammatory agents, steroids, antibiotics, analgesic agents, chemotherapeutic agents, bone morphogenetic protein, demineralized bone matrix, collagen, growth factors, autogenetic bone marrow, progenitor cells, calcium sulfate, immo suppressants, fibrin, osteoinductive materials, apatite compositions, fetal cells, stem cells, enzymes, proteins, hormones, and germicides. The mesh pouch 230 may further include or be filled with a gelatin which may contain a pharmaceutical agent 232. The gelatin inside the mesh pouch 230 may slowly osmotically leak out into the surrounding tissue.

The mesh pouch 230 may also include an adhesive to bond the mesh pouch 230 to the implantation site. Such adhesives may include cyanoacrylate adhesives, hydrogel adhesives, monomer and polymer adhesives, fibrin, polysaccharide, Indermil® or any other biocompatible adhesive. Alternatively, the mesh pouch 230 may be intracorporeally welded to the treatment to the treatment site, using surgical welding techniques.

A mesh pouch 230 filled with one or more pharmaceutical agents 232 may form a drug cocktail implant. The pharmaceutical agents 232 selected to be inserted within the mesh pouch 230 may be specifically tailored to the needs of the patient. The mesh pouch 230 may be filled outside or within the patient. Once placed within the body, the pharmaceutical agents 232 may slowly dissolve and exit the pouch 230 through an osmotic member to reach the surrounding tissue.

Referring to FIG. 35, the mesh material 220 can be positioned at least partially about a medical implant 234. The medical implant 234 can be a spacer or sponge. A pharmaceutical agent 232 can be is incorporated in the medical implant 234, for insertion into the treatment site. The pharmaceutical agent 232 seeps from the medical implant 234 to the surrounding tissue.

The medical implant 234 can be a biodegradable implant. The biodegradable implant 234 hydrophilically reacts to release the pharmaceutical agent. The biodegradable implant 234 is made of a biodegradable polymer, polyactic acid (“PLA”), polyglycolic acid (“PGA”), and copolymers thereof collagen, cellulose, fibrin, autograph, allograph, or other cellular based compounds. The pharmaceutical agents 232 may be bonded to the biodegradable implant by coating, mixing, or bonding techniques as previously described.

In another embodiment, the medical system provides a medical implant having a fillable cavity therein. The cavity is filled with a pharmaceutical agent just prior to insertion into the body of the patient. This allows the medical implant to be specifically tailored for the patient. The medical implant is used in a sterile, operating room environment to secure at least two layers of human body tissue together. The two layers of human body tissue are approximated and held against movement relative to each other with optional use of a suture which passes through the medical implant.

Referring to FIGS. 36-38, in an embodiment, the medical implant 300 includes first and second sections 302 and 304 formed separately from each other. The first section 302 includes a main body 306 having a closed wall portion defining a cavity 308 therein. The main body 306 includes a first open end portion 310 through which a pharmaceutical agent 12 may be disposed within the cavity 308. The pharmaceutical agent 12 is disposed within the cavity 308 just prior to insertion into the body of the patient. This allows the medical implant to be specifically tailored for the patient. The second, closed end 316 of the main body 306 includes a passage 318 through which a suture may be passed.

The second section 304 is a conical tip having an extended portion 320 configured to be fitted into the first end portion 310 of the main body 306. The extended portion 320 is sized to snuggly fit in the open end portion 310 of the main body 306, securing the second section 304 to the first section 302. The second section 304 covers the cavity 308, sealing in the pharmaceutical agent 12. The second section 304 may be bonded to the first section 302 utilizing an adhesive material and/or external energy source as described above.

Referring also to FIG. 39, the extended portion 320 can included a radial extension 322. A radial slot 324 can be configured about an inner surface of the first end portion 310 of the main body 306, such that upon insertion of the extended portion 320 into the first end portion 310, the radial extension 322 is engaged within the radial slot 324 securing the second section 304 to the first section 302.

The main body 306 of the first section 302 includes threads 326, allowing the medical implant 300 to be screwed into or through a first tissue layer, for example, bone, cartilage, ligaments, tendons, etc. The second end 316 of the main body 306 can have a hex-head configuration, which can be engaged by a surgical tool to screw the medical implant 300 into the tissue layer. The suture can be threaded through the passage 318 on the second end 316 of the first section 302 and the second tissue layer. The suture is tightened, securing the second tissue layer to the first tissue layer.

The pharmaceutical agent 12 is released by eluting through the first and/or second sections 302 and 304, wherein the elution rate is dependent of the thickness of the first and second sections 302 and 304. For example, the first and second sections 302 and 304 can have a uniform thickness allowing for uniform elution rate. Alternatively, the first and second sections 302 and 304 can have a variable thickness, allowing for a varying elution rate.

Alternatively, the first and second sections 302 and 304 may be made of a degradable material. The pharmaceutical agent 12 is released as the first and second sections 302 and 304 degrade. The first and second sections 302 and 304 can have uniform degradation rates, allowing for uniform release of the pharmaceutical agent 12. Similarly, the first and second sections 302 and 304 can have a variable degradation rate, allowing for a varying rate of release of the pharmaceutical agent 12.

The biodegradable first and second sections 302 and 304 may include resorbable polymer such as polyactic acid (“PLA”), polyglycolic acid (“PGA”), and copolymers thereof. The degradation rate of the biodegradable first and second sections 302 and 304 can be controlled by the ratio of PLA to PGA, or by the thickness or density of the coating. Additionally, the biodegradable first and second sections 302 and 304 may also include collagen, cellulose, fibrin, or other cellular based compounds.

In an embodiment the first and/or second section 302 and 304 of the medical implant 300 are formed of a rigid open cell material. The open cell material provides cavities through which the pharmaceutical agent 12 can be released. Alternatively, where the medical implant 360 is inserted into bone, bone can grow through the open cell material into the medical device.

The pharmaceutical agent 12 in the cavity 308 can include a bone growth inducing material. The growth of bone through the medical implant 300 is promoted by the bone growth inducing material. The bone growth inducing material in the cavity 308 may be any of many known bone morphogenic proteins and osteoinductive materials. For example, apatite compositions with collagen may be utilized. Demineralized bone powder may also be utilized. Regardless of which of the known bone growth inducing materials are selected, the presence of the bone growth promoting material in the cavity will promote a growth of bone through openings in the porous medical implant 300.

It is contemplated that the medical implant 300 may be coated with a material which promotes the growth of bone. The cells in the medical implant 300 may be at least partially filled with bone growth promoting material. The bone growth promoting materials may be bone morphogenic proteins and other osteoinductive materials.

All references cited herein are expressly incorporated by reference in their entirety. In addition, unless mention was made above to the contrary, it should be noted that all of the accompanying drawings are not to scale. There are many different features to the present invention and it is contemplated that these features may be used together or separately. Thus, the invention should not be limited to any particular combination of features or to a particular application of the invention. Further, it should be understood that variations and modifications within the spirit and scope of the invention might occur to those skilled in the art to which the invention pertains. Accordingly, all expedient modifications readily attainable by one versed in the art from the disclosure set forth herein that are within scope and spirit of the present invention are to be included as further embodiments of the present invention. The scope of the present invention is accordingly defined as set forth in the appended claims.

What is claimed is:

1. A medical system for the administration of a pharmaceutical agent in vivo comprising:
a medical implant positionable in a body of a patient and including the pharmaceutical agent and a reactive coating thereon, wherein the reactive coating controls the release of the pharmaceutical agent; and
an energy unit for transmitting an energy signal to the reactive coating, wherein the reactive coating reacts to the energy signal to increase the release rate of the pharmaceutical agent.

2. The medical system of claim 1, further comprising a plurality of reactive coatings, wherein the plurality of reactive coatings are layered.

3. The medical system of claim 2, wherein the pharmaceutical agent is interposed between each of the layers of the plurality of reactive coatings.

4. The medical system of claim 3, wherein at least one different pharmaceutical agent is disposed between at least one of the layers of the plurality of reactive coatings.

5. The medical system of claim 1, wherein the pharmaceutical agent is mixed within the reactive coating.

6. The medical system of claim 5, further comprising a plurality of reactive coatings, wherein the plurality of reactive coatings are layered.

7. The medical system of claim 6, wherein at least one different pharmaceutical agent is mixed within at least one of the layers of the plurality of reactive coatings.

8. The medical system of claim 1, wherein the reactive coating is biodegradable.

9. The medical system of claim 8, wherein the medical implant is biodegradable.

10. The medical system of claim 9, wherein the energy signal increases the degradation rate of the biodegradable coating.

11. The medical system of claim 9, further comprising a plurality of biodegradable coatings, wherein the plurality of biodegradable coatings are layered.

12. The medical system of claim 11, wherein the pharmaceutical agent is interposed between each of the biodegradable layers.

13. The medical system of claim 12, wherein at least one different pharmaceutical agent is disposed between at least one of the biodegradable layers.

14. The medical system of claim 11, wherein the pharmaceutical agent is mixed within each of the biodegradable layers.

15. The medical system of claim 14, wherein at least one different pharmaceutical agent is mixed within at least one of the biodegradable layers.

16. The medical system of claim 11, wherein the energy signal separates at least one biodegradable coating layer from the medical implant to release the pharmaceutical agent.

17. The medical system of claim 1, wherein the reactive coating is a porous coating and the application of the energy signal increases the uniform pore size to increase the release rate of the pharmaceutical agent.

18. The medical system of claim 17, wherein the porous coating has a uniform pore size.

19. The medical system of claim 17, where the discontinuation of the energy signal returns the uniform pore size to an original pore size.

20. The medical system of claim 17, wherein the porous coating has a plurality of different pore sizes.

21. The medical system of claim 20, where each of the plurality of different pore sizes is attuned to react to a different energy signal frequency.

22. The medical system of claim 21 wherein the energy signal selectively increases at least one of the plurality of pore sizes to selectively release the pharmaceutical agent.

23. The medical system of claim 1, wherein the reactive coating substantially prevents release of the pharmaceutical agent and the energy signal disrupts the coating thereby allowing elution of the pharmaceutical agent through the coating.

24. The medical system of claim 23, further comprising a second coating positioned between the reactive coating and the medical implant, wherein the second coating is impregnated with the pharmaceutical agent.

25. The medical system of claim 23, wherein the energy signal creates fissures in the reactive coating.

26. The medical system of claim 23, wherein the medical implant has a first coefficient of thermal expansion and the coating has a second coefficient of thermal expansion.

27. The medical system of claim 26, wherein the first coefficient of thermal expansion is greater then the second coefficient of thermal expansion.

28. The medical system of claim 23, wherein the coating includes a non-resorbable polymer.

29. The medical system of claim 28, wherein the pharmaceutical agent is mixed within the non-resorbable polymer.

30. The medical system of claim 1, wherein the medical implant is made of a biological material.

31. The medical system of claim 1, wherein the energy unit is an external energy unit positioned on a skin portion of the body of the patient proximal to the medical implant.

32. The medical system of claim 1, wherein the energy unit is an internal energy unit positionable within the body of the patient.

33. The medical system of claim 32, wherein the internal energy unit in operable connected to an external power source.

34. The medical system of claim 33, wherein the internal energy unit includes an internal power source.

35. The medical system of claim 34, wherein the internal energy unit includes a control unit.

36. Then medical system of claim 35, wherein the control unit is programmed to activate the internal energy unit at set time intervals.

37. The medical system of claim 35, where the control unit activates the internal energy unit in response to an external signal.

38. The medical system of claim 34, wherein the internal power source includes a rechargeable battery.

39. The medical system of claim 1, wherein the external energy unit transmits an energy signal selected from the group consisting of radio frequency (RF), magnetic, electro magnetic (EM), acoustic, microwave, thermal, vibratory, radiation, extracorporeal shockwave (ESW) energies, and combination thereof.

40. The medical system of claim 1, wherein the pharmaceutical agent is selected from a group consisting of a drug, therapeutic agent, and biological agent.

41. The medical system of claim 1, wherein the medical implant is selected from a group consisting of a stent, hip replacement, knee replacement, spinal implant, tissue, scaffold, biological implants, graft, tissue graft, screws, plate, rods, and prosthetic device.

42. The medical system of claim 1, wherein the reactive coating comprise a plurality of capsules bonded together.

43. The medical system of claim 42, wherein the pharmaceutical agent is disposed within each of the capsules.

44. The medical system of claim 42, wherein the capsules have a uniform size.

45. The medical system of claim 42, wherein the capsules have a plurality of different sizes.

46. The medical system of claim 45, wherein a different pharmaceutical agent is disposed within each of the different capsule sizes.

47. The medical system of claim 45, where each of the plurality of different capsules sizes is attuned to react to a different energy signal frequency or wavelength.

48. The medical system of claim 42, wherein the plurality of capsules are made of a biodegradable material.

49. A method of releasing a pharmaceutical agent from an implantable device comprising the steps of:
providing an implantable device impregnated or coated with a pharmaceutical agent, the implantable device having a barrier substantially limiting release of the pharmaceutical agent therethrough;
implanting the implantable device in tissue in a body; and
directing energy at the implantable device,
wherein the energy directed at the implantable device disrupts the barrier thereby allowing elution of the pharmaceutical agent through the barrier to the tissue.

50. The method of claim 4S, wherein the energy directed at the implantable device creates fissures in the barrier.

51. The method of claim 50, wherein the barrier is a first coating covering the implantable device.

52. The method of claim 51, further comprising a second coating positioned between the first coating and the implantable device and wherein the pharmaceutical agent is found in the second coating.

53. The method of claim 48, wherein the implantable device is made of an implant material having a first thermal expansion coefficient and the barrier is made of a barrier material having a second thermal expansion coefficient and wherein the energy directed at the implantable device heats the implantable device and barrier.

54. The method of claim 53, wherein the barrier material includes a non-resorbable polymer.

55. The method of claim 54, wherein the pharmaceutical agent is mixed with the non-resorbable polymer.

56. The method of claim 49, wherein the energy directed at the implantable device is selected from the group consisting of radio frequency (RF), magnetic, electro magnetic (EM), acoustic, microwave, thermal, vibratory, radiation, extracorporeal shockwave (ESW) energies, and combination thereof.

57. A medical implant for the administration of a pharmaceutical agent in vivo comprising:
a body portion including a reservoir therein;
a pharmaceutical agent disposed within the reservoir; and
a cover portion attachable to the body portion, covering the reservoir and the pharmaceutical agent.

58. The medical implant of claim 57, wherein the body portion comprises a plurality of reservoirs, wherein each of the reservoirs contains the pharmaceutical agent.

59. The medical implant of claim 58, where each of the reservoirs contains a different pharmaceutical agent.

60. The medical implant of claim 57, wherein the pharmaceutical agent is selected from a group consisting of a drug, therapeutic agent, and biological agent.

61. The medical implant of claim 57, wherein the body and cover portions are made of a biodegradable material.

62. The medical implant of claim 61, wherein the degradation rate of the body and cover portions controls the release of the pharmaceutical agent.

63. The medical implant of claim 57, where the thickness of the body and cover portions controls the release of the pharmaceutical agent.

64. A method for administration of a pharmaceutical agent into a patient, comprising:
selecting a medical implant for insertion into the patient;
selecting the pharmaceutical agent;
dispensing the pharmaceutical agent into the medical implant prior to insertion of the medical implant into the patient; and
inserting the medical implant into the body of the patient.

65. The method of claim 64, wherein the medical implant comprises:
a body portion including a reservoir therein; and
a cover portion attachable to the body portion, covering the reservoir

66. The method of claim 65, wherein dispensing the pharmaceutical agent comprises;
positioning the pharmaceutical agent in the reservoir of the body portion, and
attaching the cover portion to the body portion, covering the reservoir and the pharmaceutical agent therein.

67. The method of claim 66 wherein the pharmaceutical agent elutes through the medical implant.
